(19) United States
(12) Patent Application Publication
(10) Pub. No.: US 2005/0192264 A1
(43) Pub. Date: Sep. 1, 2005

(54) Slow release steroid composition

(76) Inventor: Philip Leslie Penfold, Australian Capital Territory (AU)

Correspondence Address: FROMMER LAWRENCE & HAUG, 745 FIFTH AVENUE- 10TH FL., NEW YORK, NY, 10151, US

(21) Appl. No.: 11/051,028

(22) Filed: Feb. 4, 2005

(30) Foreign Application Priority Data
Feb. 4, 2004 (AU) 2004900546
Sep. 10, 2004 (AU) 2004905195
Oct. 25, 2004 (AU) 2004906125
Jan. 21, 2005 (AU) 2005900253

(51) Int. Cl. A61K 31/573

(52) U.S. Cl. 514/179

(57) ABSTRACT

A pharmaceutically acceptable composition comprising an anti-inflammatory steroid or pharmaceutically acceptable salt thereof, which exists in varying crystal and crystal composite sizes wherein the proportion of crystals and crystal composites above 20 μm in size in the composition is greater than the proportion of crystals and crystal composites under 20 μm in size.

### INCORPORATION BY REFERENCE

This application claims benefit of Australian Provisional Patent Application Nos. 2004900546 filed 4 Feb. 2004, 2004905195 filed 10 Sep. 2004, 2004906125 filed 25 Oct. 2004 and 2005900253 filed 21 Jan. 2005.

The foregoing applications, and all documents cited therein, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention relates to the treatment of degenerative retinopathies which are amenable to treatment with an anti-inflammatory steroid present in a particulate form, the crystal size and distribution of which are determinable and selectable. In particular, it relates to the use of a range of crystal sizes of triamcinolone and in particular triamcinolone acetonide used to treat inflammatory eye conditions.

### BACKGROUND ART

The present inventor was the co-inventor of U.S. Pat. No. 5,770,589 to Billson and Penfold (“U.S. '589”), which was filed as U.S. application Ser. No. 08/586,750, and is incorporated herein in its entirety by reference. U.S. '589 provides a method for the treatment of age related macular degeneration in a patient and may comprise administering by intravitreal injection to the patient, an effective amount in depot form of an anti-inflammatory steroid which is preferably sparingly soluble in the vitreous. Preferred steroids used in the method described in U.S. '589 may include triamcinolone acetonide (TA).

The present inventor is also co-inventor of Australian Patent No. 769,671 to Gillies, Penfold and Billson (AU '671), which was filed as Australian Patent Application No. 46732/99 and is directed to the prophylaxis of neovascularisation by intravitreal injection of an anti-inflammatory steroid into an eye which has been identified as having a high risk of developing choroidal neovascularisation.

Preferred steroids used in the method of AU '671 may include triamcinolone acetonide and fluocinolone acetonide.

While the methods of treatment presented in these patent specifications have been encouraging with respect to, for example, both pilot studies and subsequent continuing clinical trials, there is a need for further improvements to methods for treating above-mentioned ocular conditions, including, but not limited to, degenerative retinopathies, ocular neovascularisation, and inflammatory eye conditions.

One such desirable improvement, especially in view of certain indications and disease circumstances, would be to prolong the therapeutic effect of anti-inflammatory drugs. For example, in the case of triamcinolone acetonide administration, Massin et al. have found that in most cases the beneficial effects of intravitreal injection of the usual dose (4 mg) of triamcinolone acetonide in the treatment of clinically significant macular edema and other retinal diseases is diminished after approximately three months: e.g., Massin P, et al. (2004) “Intravitreal triamcinolone acetonide for diabetic diffuse macular edema: preliminary results of a prospective controlled trial” Ophthalmology 111(2): 218-24. Massin et al. measures the beneficial effects of triamcinolone acetonide based on measurements of visual acuity. For example, optical coherence tomography as used in the case of macular oedema shows that the peak efficacy of triamcinolone acetonide administered as a single intravitreal injection is achieved approximately six weeks after the single intravitreal injection, which is followed by a decline in visual acuity and a thickening of the macula. Another study showed that the mean elimination half-life of triamcinolone acetonide administered as a single intravitreal injection was 18.6 days in nonvitrectomized patients: e.g., Beer P M, et al. (2003) “Intraocular concentration and pharmacokinetics of triamcinolone acetonide after a single intravitreal injection” Ophthalmology 110(4): 681-6. Given the assumption that all of the triamcinolone acetonide will be eliminated after about five half-lives, triamcinolone acetonide should persist at measurable concentrations in the vitreous humour for about three months (93±28 days) in patients who have not undergone vitrectomy. While a three month dwell time for anti-inflammatory steroids like triamcinolone acetonide may be reasonable for certain disease circumstances, many patients would benefit from even longer therapeutically effective dwell times because there would be as lower frequency of injections and lower risk of complications relating to intraocular injection.

A number of strategies have been advanced to prolong the effect of anti-inflammatory drugs. One approach that has been used in the case of has been to simply increase the dosage used (up to 25 mg): e.g., Jonas J B, et al. (2004) “Duration of the effect of intravitreal triamcinolone as treatment for diffuse diabetic macular edema” Am J Ophthalmol. 138(1):158-60 and Jonas J B, (2004) “Intraocular availability of triamcinolone acetonide after intravitreal injection” Am J Ophthalmol. 137(3):560-2. While this may prolong the duration of triamcinolone acetonide's therapeutic effect, it also may increase the incidence and severity of complications, especially elevated steroid-induced intraocular pressure, e.g., Degenring R F, et al. (2004) “Intraocular triamcinolone for diffuse diabetic macular edema” Ophthalmologe. 101(3):251-4.

Another possible approach that could be used to prolong the effect of anti-inflammatory drugs is to use a sustained release vehicle, which has been used effectively with other intravitreal steroid preparations. However, one major disadvantage of such an approach is that, due to its increased level of complexity, ultimate approval for a drug formulated with a sustained release vehicle may be delayed by a regulatory agency, such as the US Food and Drug Administration (US FDA).

A third approach that may increase the therapeutically effective dwell time of an anti-inflammatory drug may be to vary the particle size. However, this approach is not settled with respect to many drugs, such as drugs for treating eye diseases. In fact, some drugs appear not to show any correlation between particle size and the therapeutically effective dwell time in the vitreous. For example, a recent study reports that two triamcinolone acetonide preparations with significantly different median particle sizes (4 microns and 17.3 microns) had essentially the same half life of the drug in the vitreous:, e.g., Robinson et al., “Preclinical Evaluation of a Triamcinolone Acetonide Preservation Free (TAC-PF) Formulation for Intravitreal Injection,” Association for Research in Vision and Ophthalmology (ARVO), 2004.

Another problem associated with varying a drug's particle size to control the therapeutically effective dwell time relates to the inventor's discovery that certain drug particles, especially anti-inflammatory steroids at particle sizes below 0.5 um and up to about 1 um, tend to cause blockages to delivery needles through which they are administered. Typically, when administering drugs to the vitreous, ophthalmologists like to use as fine (e.g. small diameter) a needle as possible to penetrate the outer structures of the eye such as the sclera. Intuitively, the use of smaller-diameter needles should also lead to the choice of smaller drug particles, such as smaller particles of anti-inflammatory steroids, to be injected in view of the logical expectation that larger particles would tend to block the needle during injection of the steroid into the eye. However, the inventor has discovered that particles below 0.5 μm up to about 1 μm tend to block a needle by a process called “flocculation” or “compaction.”

Thus, there exists a need to develop a means for prolonging the therapeutic effects of anti-inflammatory steroids, such as triamcinolone acetonide, after a single intravitreal injection, while ameliorating the problems attendant with the delivery of such compounds.

Citation or identification of any document in this application is not an admission that such document is available as prior art to the present invention.

### SUMMARY OF THE INVENTION

Research conducted to address the problems attendant with the prior art has revealed that anti-inflammatory steroid drugs, such as triamcinolone acetonide, which when prepared as micronised crystals, are often fused, aggregated or flocculated together in the form of crystal composites. Crystal composites, while having the appearance of larger particles are actually formed of smaller crystals (for example, see FIG. 1). In addition, the crystal composites have dissolution rates that are similar to the dissolution rates of smaller crystals. Thus, in accordance with one principle of the present invention, a key to increasing the therapeutically effective intravitreal dwell time of an anti-inflammatory steroid, such as, for example, triamcinolone acetonide, without increasing the total concentration of the drug in a therapeutic formulation is to increase the proportion of larger-sized crystals of the anti-inflammatory steroid while decreasing the proportion of crystal composites of the steroid. Accordingly, the dissolution rate of an anti-inflammatory steroid drug, such as, for example, triamcinolone acetonide, may be decreased (i.e. longer dissolution time) by increasing the relative proportion of large-sized drug crystals in a therapeutic composition or formulation. The larger-sized drug crystals have a decreased surface area to volume relationship relative to crystal composites, and as such, have a lower rate of dissolution.

According to a first aspect of this invention, there is provided a pharmaceutically acceptable composition comprising an anti-inflammatory steroid or pharmaceutically acceptable salt thereof, which exists in varying crystal and crystal composite sizes and wherein the proportion of crystals and crystal composites above about 20 μm in size in the composition is greater than the proportion of crystals and crystal composites under about 20 μm in size. Desirably, the composition will include crystals within the size range of about 50 μm to about 600 μm. Preferably, the proportion of crystals in the size range of about 50 μm to about 600 μm will be greater than the proportion of similarly sized crystal composites.

According to a second aspect of this invention, there is provided a pharmaceutically acceptable composition comprising an anti-inflammatory steroid or pharmaceutically acceptable salt thereof which is present in the form of crystals and crystal composites of varying sizes and wherein said crystals are concentrated in the size ranges of about 0.5 μm to about 40 μm and about 50 μm to about 600 μm. Preferably the crystals are more concentrated than the crystal composites in the size ranges of about 0.5 μm to about 40 μm and about 50 μm to about 600 μm. Even more preferably the proportion of crystals in the size ranges of about 50 μm to about 600 μm is greater than that provided in the about 0.5 μm to about 40 μm size range.

According to a third aspect of this invention, there is provided a method of preparing a pharmaceutically acceptable triamcinolone composition which has an improved therapeutically effective dwell time in the vitreous of a patient, said method comprising the steps of: increasing the concentration of crystals, as compared to crystal composites, in the composition.

According to a fourth aspect of this invention, there is provided a method of preparing a pharmaceutically acceptable triamcinolone composition which has an improved therapeutically effective dwell time in the vitreous in a patient, said method comprising the steps of: increasing the proportion of crystals of a size of about 50 μm to about 600 μm in a given triamcinolone preparation compared to the proportion of about 50 μm to about 600 μm crystal composites.

According to a fifth aspect of this invention, there is provided a method of preparing a pharmaceutically acceptable composition which has an improved therapeutically effective dwell time in the vitreous of a patient, said method comprising the steps of: selecting triamcinolone crystals in the size range of about 50 μm to about 600 μm from a triamcinolone composition comprising both crystals and crystal composites. The method may include the additional step of adding said range of crystals to an ophthalmologically acceptable carrier, diluent and/or excipient.

According to a sixth aspect of this invention, there is provided a pharmaceutically acceptable composition prepared according to any one of the methods described in the third, fourth or fifth aspects of the invention.

According to a seventh aspect of this invention, there is provided a method of treating inflammatory eye conditions in a patient requiring said treatment, said method comprising administering to or adjacent to at least an ocular tissue a pharmaceutically acceptable composition as herein disclosed or a pharmaceutically acceptable composition prepared by the method as herein disclosed.

Preferably, the anti-inflammatory steroid is a member of the triamcinolone family of compounds. In a highly preferred form of the invention the triamcinolone compound used is triamcinolone acetonide. In addition, the specification may refer to the anti-inflammatory steroids of the invention, including the triamcinolone acetonide of the invention, as “active compound” or the “therapeutic” compounds of the invention. It is preferred that the pharmaceutically acceptable compositions of the present invention are delivered to the eye by intravitreal injection or topical application. It will be appreciated, however, that the mode of delivery, i.e. the delivery route or method, is not limited to intravitreal injection or topical application, but rather may include any suitable method known or used by one of ordinary skill in the art.

Other objects, features, and advantages of the instant invention, in its details as seen from the above, and from the following description when considered in light of the appended claims.

### BRIEF DESCRIPTION OF THE DRAWINGS

Comprehension of the invention is facilitated by reading the following detailed description, in conjunction with the annexed drawings.

FIG. 1 is an electron micrograph showing particles of triamcinolone acetonide exhibiting a range of crystal sizes, with the row of 11 dots along the side of the Figure representing 4.30 μm.

FIG. 2 is an electron micrograph of a specific range of particles of triamcinolone acetonide, with the row of 11 dots along the side of the Figure representing 1.19 μm.

FIG. 3A is a histogram representation of particle size analysis of a first batch of Kenacort® A 40 triamcinolone acetonide.

FIG. 3B is a histogram representation of particle size analysis of a second batch of Kenacort® A 40 triamcinolone acetonide.

FIG. 4 is a histogram representation of particle size analysis of a batch of triamcinolone acetonide of Italian origin.

FIG. 5 is a histogram representation of particle size analysis of a batch of triamcinolone acetonide of Chinese origin.

FIG. 6 is an electron micrograph showing particles of triamcinolone acetonide exhibiting a range of crystal sizes, with the row of 11 dots at the base of the Figure representing 120 μm.

FIG. 7 is another electron micrograph showing particles of triamcinolone acetonide exhibiting a range of crystal sizes, with the row of 11 dots at the base of the Figure representing 120 μm.

FIG. 8 is an electron micrograph of non-micronised triamcinolone acetonide from Farmabios showing crystals that are chunky in shape (magnification 160×, size 100-400 μm long).

FIG. 9 is an electron micrograph of non-micronised triamcinolone acetonide from NewChem showing crystals that are needle-like in shape (magnification 160×, size 200-500 μm long).

FIG. 10 is an electron micrograph of non-micronised triamcinolone acetonide from Farmabios showing crystals that are porous (magnification 1020×, size 80 μm diameter).

FIG. 11 is an electron micrograph of non-micronised triamcinolone acetonide from NewChem showing crystals that are non-porous (magnification 2600×, size 80 μm diameter).

FIG. 12 is a graph showing the particle profile size of non-micronised triamcinolone acetonide from Farmabios and NewChem determined by laser light scattering (particle size range from 20-90 μm, error bars for average (n=3)±s.d).

FIG. 13 is a graph showing the particle size profile of micronised triamcinolone acetonide from Farmabios determined by laser light scattering (particle size range from 5-50 μm, insert shows same data on log scale for clarity).

FIG. 14 is a graph showing the dissolution of non-micronised Farmabios and NewChem triamcinolone acetonide samples at 37° C., in USP2 Apparatus with 1 mL of 2 mg/mL suspension added to 400 mL saline at t=0 (data are mean±s.d., n=3).

FIG. 15 is a graph showing a comparison of dissolution rates of Farmabios and NewChem non-micronised triamcinolone acetonide left for 8 h.

FIG. 16 is a graph showing a comparison of dissolution rates of Farmabios and NewChem non-micronised triamcinolone acetonide left for 80 h.

FIG. 17 is a graph showing the dissolution of Farmabios micronised and non-micronised triamcinolone acetonide samples over 6 hours at 37° C., in USP2 Apparatus with 1 mL of 2 mg/mL suspension added to 400 mL saline at t=0.

FIG. 18 is a graph showing the dissolution of micronised and non-micronised triamcinolone acetonide from Farmabios over 20 hours at 37° C., in USP2 Apparatus with 1 mL of 2 mg/mL suspension added to saline at t=0.

FIG. 19 is a graph showing the dissolution of Farmabios micronised and non-micronised triamcinolone acetonide samples and mixtures thereof at 37° C., in USP2 Apparatus with 1 mL of 2 mg/mL suspension added to 400 mL saline at t=0 (data are mean±s.d., n=3).

FIG. 20 is a graph showing the dissolution of Farmabios 100% micronised triamcinolone acetonide at 37° C., in USP2 Apparatus with 1 mL of 2 mg/mL suspension added to 400 mL saline at t=0 (data are mean±s.d., n=3).

FIG. 21 is a graph showing the dissolution of micronised Farmabios triamcinolone acetonide in 3% CMC Gel at 37° C. over 180 min, in USP2 Apparatus with 1 mL of 2 mg/mL suspension added to 400 mL 3% CMC in saline at t=0 (data are mean±s.d., n=3).

FIG. 22 is a graph showing the dissolution of non-micronised Farmabios and NewChem triamcinolone acetonide together with an 80:20 non-micronised: micronised mixture in 3% CMC Gel at 37° C., in USP2 Apparatus with 1 mL of 2 mg/mL suspension added to 400 mL 3% CMC in saline at t=0 (data are mean±s.d., n=3).

FIG. 23 is an illustration of a simulated eye diffusion apparatus.

FIG. 24 is a graph showing the simulated eye diffusion apparatus experiments with micronised and non-micronised triamcinolone acetonide in 1% hyaluronic acid (HA) gel at 37° C. over 14 days (data are mean±range, n=2).

FIG. 25 is a graph showing the simulated eye diffusion apparatus experiments with micronised and non-micronised triamcinolone acetonide and mixtures thereof in 1% HA gel at 37° C. over 14 days (data are mean±range, n=2).

FIG. 26 is a graph showing the particle size of three fractions obtained from sedimentation separation of 100 mg of non-micronised NewChem triamcinolone acetonide added to a 1 metre column.

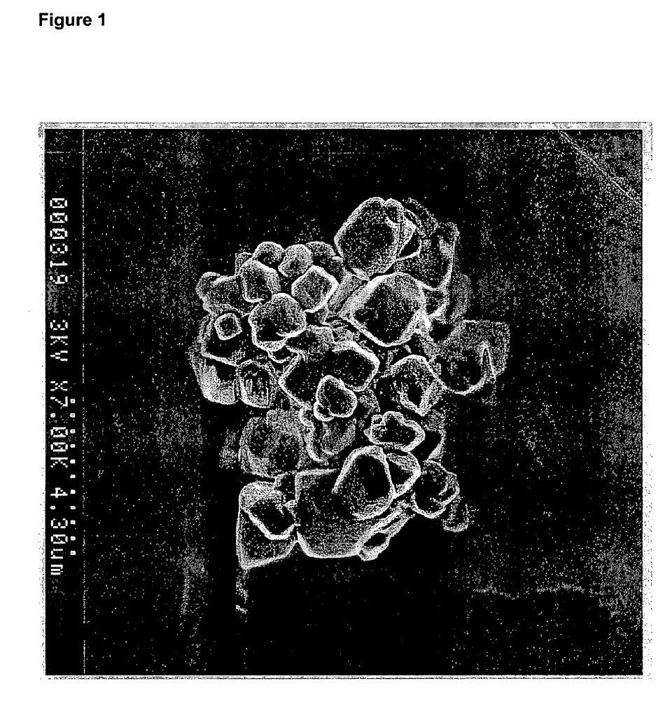

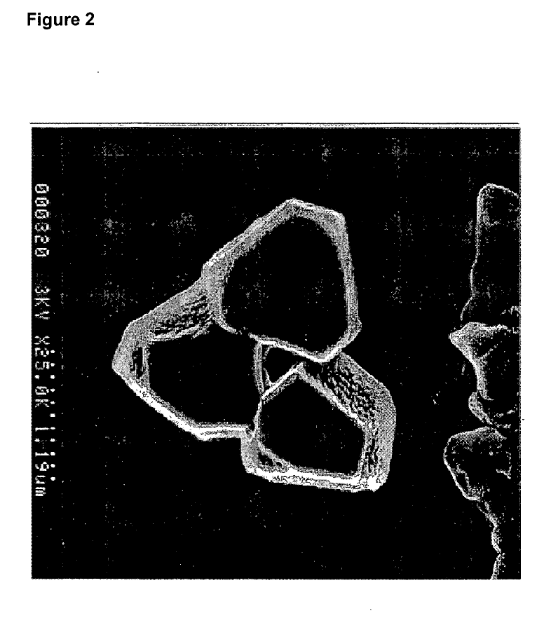

### DISCLOSURE OF THE INVENTION

General

Those skilled in the art will appreciate that the invention described herein is susceptible to variations and modifications other than those specifically described. The invention includes all such variation and modifications. The invention also includes all of the steps, features, compositions and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

The present invention is not to be limited in scope by the specific embodiments described herein, which are intended for the purpose of exemplification only. Functionally equivalent products, compositions and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the description of the invention and apply throughout. Unless otherwise defined, all other scientific and technical terms used herein have the same meaning as commonly understood to one of ordinary skill in the art to which the invention belongs.

### DETAILED DISCLOSURE OF THE INVENTION

This invention is based on the unexpected discovery that a substantially flat dissolution curve of an anti-inflammatory steroid can be achieved without changing the total drug exposure at the site of action of the active compound by increasing the crystal sizes in a given anti-inflammatory steroid preparation while deselecting for crystal composites. Crystals, as distinct from composites provide a longer lasting source of active compound in the eye.

According to a first aspect of this invention, there is provided a pharmaceutically acceptable composition comprising an anti-inflammatory steroid, such as, for example triamcinolone acetonide, or pharmaceutically acceptable salt thereof, which exists in varying crystal and crystal composite sizes wherein the proportion of crystals and crystal composites above about 20 μm in size in the composition is greater than the proportion of crystals and crystal composites under about 20 μm in size. Desirably, the composition will include crystals within a size range of about 50 μm to about 600 μm. Preferably, the proportion of crystals in the size range of about 50 μm to about 600 μm will be greater than the proportion of similarly sized crystal composites.

According to a second aspect of this invention, there is provided a pharmaceutically acceptable composition comprising an anti-inflammatory steroid, such as, for example triamcinolone acetonide, or pharmaceutically acceptable salt thereof which is present in the form of crystals and crystal composites of varying sizes and wherein, said crystals are concentrated in the size ranges of of about 0.5 μm to about 40 μm and of about 50 μm to about 600 μm. Preferably, the crystals are more concentrated than the crystal composites in the size ranges of about 0.5 μm to about 40 μm and about 50 μm to about 600 μm. Even more preferably the proportion of crystals in the size ranges of about 50 μm to about 600 μm is greater than that provided in the about 0.5 μm to about 40 μm size range.

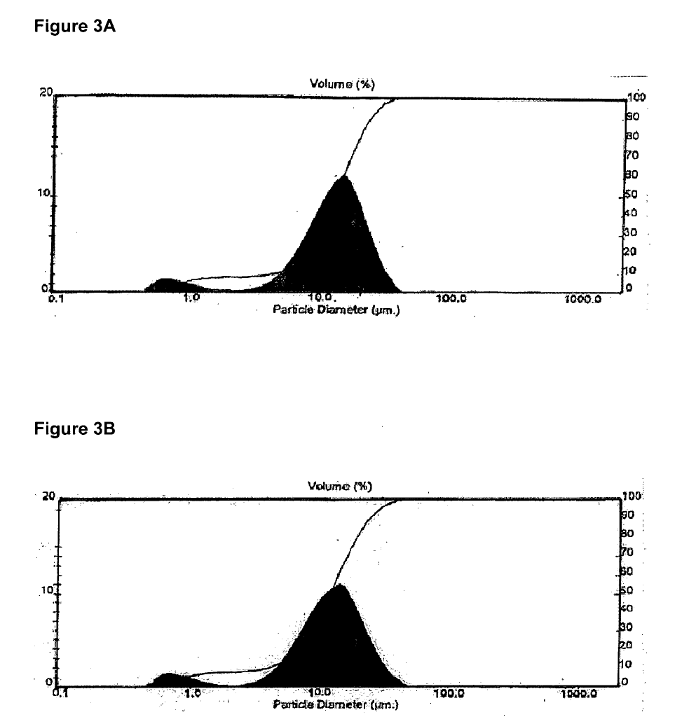

As used herein the term “crystal composite” includes both crystals and non-crystals that are aggregated, fused or in some other way bound together. The phrase will include composites that remain aggregated after passage through a syringe needle (such as, but not limited to, a 27 gauge needle). It can be seen by reference to light scatter measurements (Table 6 and 7, FIGS. 3A, 3B, 4 and 5) that crystal sizes range from about 0.5 μm in Kenacort® A 40 to about 600 μm in NewChem non-micronised material.

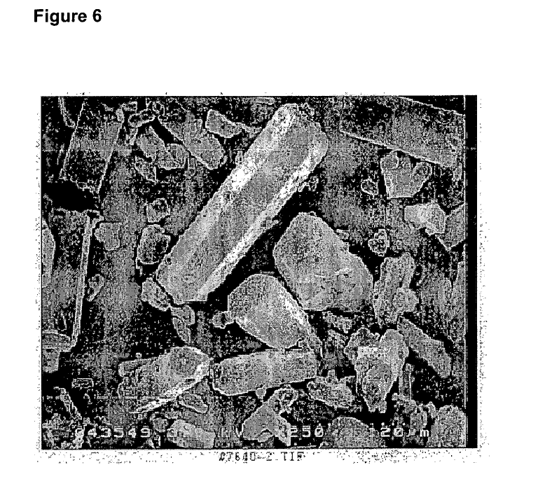

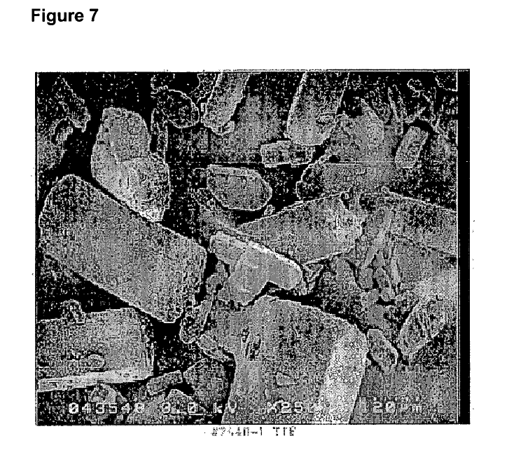

As used herein the term “crystal” in the context of this invention has as its normal meaning a solid body having a characteristic internal structure and enclosed by symmetrically arranged planar surfaces, intersecting at definite and characteristic angles. Ordinarily a crystal will not be a composite of smaller crystals. However, a single large crystal may have much smaller crystals attached to it. When a crystal is present in such a form, it will not be considered a crystal composite. FIGS. 6 and 7 show, for example, that by scanning electron microscopy, the size of crystals varies but crystal sizes of greater than 120 μm can be observed (the series of 11 dots at the base of FIG. 6 represents 120 μm, and the series of 11 dots at the base of FIG. 7 represents 120 μm).

Crystals are said to be concentrated in a preparation where the preparation has been modified to increase the crystal content in a particular size range. This may be achieved by selecting crystals of a particular size and then combining those crystals with another preparation or by using the crystals as a preparation. Methods for selecting crystals of a particular size will be understood by one of ordinary skill in the art.

Crystal sizes within the ranges mentioned will vary depending on the dissolution time required and the longevity of action required of the anti-inflammatory steroid in or adjacent to the ocular tissue to be treated. Preferably, crystals in the upper size range will vary between about 50 μm to about 600 μm, about 60 μm to about 500 μm, about 70 μm to about 400 μm, about 80 μm to about 300 μm, about 90 μm to about 250 μm or about 100 μm to about 200 μm. Where the lower size range is included, the crystals will be in the range of between about 1 μm to about 40 μm, about 5 μm to about 35 μm, about 10 μm to about 30 μm, about 15 μm to about 25 μm or about 20 μm to about 22 Atm.

Anti-inflammatory steroids that may be utilised in this invention will be those that are capable of being prepared in either crystal or crystal composites size ranges as herein described, which have an anti-inflammatory action and which are suitable for use in the treatment of inflammatory disorders in the ocular region. Preferred anti-inflammatory steroids may include, for example, 11-substituted-16α,17α-substituted methylenedioxy steroids of the formula: 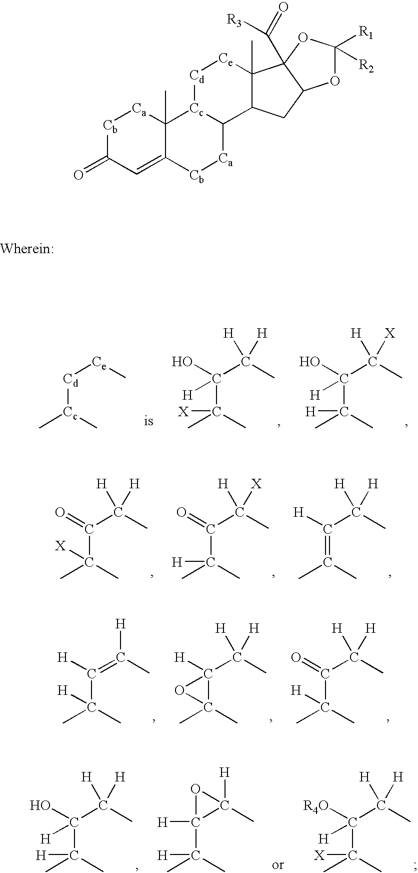
R₁ and R₂ are hydrogen or alkyl; 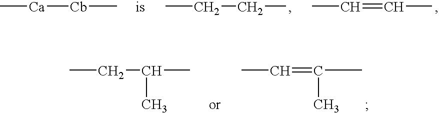
R₃ is methyl, hydroxymethyl or methylaminoalkylenecarbonyloxymethyl, alkylcarbonyloxymethyl, or phenylaminoalkylenecarbonyloxymethyl;
R₄ is alkanoyl; and
X is a halogen.

More preferred are compounds of the formula: 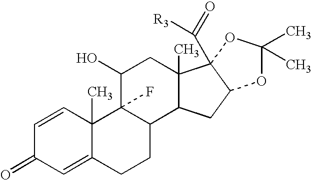
wherein R₃ is hydroxymethyl, phenylcarbonylaminoisopropylcarbonyloxymethyl, or 2,2-dimethylpropylcarbonyloxymethyl.

One preferred steroid is crystalline 9-fluoro-11,21-dihydroxy-16,17-[1-methylethylidinebis(oxy)]pregna-1,4-diene-3,20-dione:

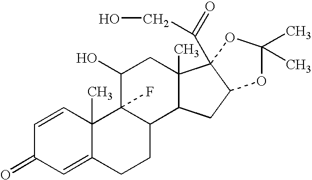

This compound, also known by its generic name as triamcinolone acetonide, is suitably prepared by known methods such as those disclosed in Fried et al. (1958) J. Am. Chem. Soc. 80, 2338 (1958); U.S. Pat. No. 2,990,401; U.S. Pat. No. 3,048,581 or U.S. Pat. No. 3,035,050 each of which is expressly herein incorporated by reference.

In order to ensure that the active compound (i.e. the anti-inflammatory steroids described herein) of the present invention is present in the form of crystals, rather than as crystal composites, the composites are preferably disrupted prior to preparation of the composition of the invention. Anti-inflammatory steroid crystal composites may be disrupted by a variety of methods known in the art such as sonication and micronisation (defined as particles <30 μm). Sonication may be used to break down crystal composites when used for short periods (eg 30 sec) or may be used to fracture crystals when used for more extended periods. Crystal sizes may additionally be influenced by re-crystallisation/growth, gamma-irradiation and high temperature (eg autoclaving). Particles or crystals may be fractionated also by methods known in the art, such as centrifugation on a density gradient of inert carrier, selective filtration or dry sieving; or other methods known in the art of fractionating microscopic material.

It has been found that particles and crystals below about 0.5 μm to about 1 μm tend to block a needle by the process of flocculation or compaction if the compositions are delivered by injection. In addition, these smaller particles dissolve rapidly in the vitreous, thereby disappearing from the intraocular environment more quickly than larger particles. In contrast, particles of about 1 μm to about 12 μm remain in the intraocular environment for a sufficient period of time to give a residual effect and do not tend to block a needle when being delivered from a syringe.

Where crystals are concentrated in the size ranges of about 0.5 μm to about 40 μm and about 50 μm to about 600 μm the characteristics of the composition may be varied by reducing or increasing the relative weight per volume of particles in the lower size range relative to the weight per volume of particles in the upper size range. By varying such characteristics the skilled artisan can develop compositions with differing dwell time in the vitreous which may be important depending on the ailment to be treated and the longevity required for such treatment. For example, the weight per volume ratio of lower size range crystals to upper size range crystals may be about 1:1, 1:2, 2:1, 1:3, 3:1, 2:3, 3:2, 1:4, 4:1, 3:4, 4:3, 1:5, 5:1, 2:5, 5:2, 3:5, 5:3, 4:5, 5:4, 1:6, 6:1, 5:6, 6:5.

In an alternative way of looking at the invention the percentage of particles or crystals in the lower size range may be greater than 1%, 5%, 10% 15%, 20%, 25%, 30%, 35%, 40%, 45%, 50%, 55%, 60%, 65%, 70%, 75%, 80%, 85%, 90%, 95%, 99% w/v with the reciprocal value being in the upper range. Similarly, the percentage of particles or crystals in the upper size range may be greater than 1%, 5%, 10% 15%, 20%, 25%, 30%, 35%, 40%, 45%, 50%, 55%, 60%, 65%, 70%, 75%, 80%, 85%, 90%, 95%, 99% w/v with the reciprocal value being provided in the lower range.

Depending on the distribution of the crystals in the about 50 μm to about 600 μm fraction compared to the about 0.5 μm to about 40 μm fraction, dwell time of the formulation can be substantially improved. As used herein dwell time will refer to the time that the therapeutic composition can achieve a therapeutic effect against the ailment that it is used to treat.

Improvements in dwell time generally increase as the proportion of crystals in the about 50 μm to about 600 μm fraction increases, however a balance needs to be struck between long dwell time and achieving a therapeutic effect. Where that balanced is struck can depend on the longevity of anti-inflammatory effect required in the eye and the ailment being treated. Formulations prepared according to the invention will preferably have dwell times in excess of at least 2 months. More preferably the dwell time will be greater than 3 months, with dwell times of greater than 4, 5, 6, 7 to 12 months being achievable and highly desired.

By way of illustration the composition of the present invention may comprise 25% w/v of crystals of about 0.5 μm to about 40 μm and 75% w/v of crystals of about 50 μm to about 600 μm. Preferably the composition may comprise about 20% w/v of crystals of about 0.5 μm to about 40 μm and about 80% w/v of crystals of about 50 μm to about 600 μm. These proportions allow an initial dose of the active compound, followed by longer term maintenance of a substantially flat dissolution curve of the anti-inflammatory steroid and increased dwell time. Alternatively, about 50% w/v of crystals of about 0.5 μm to about 40 μm and about 50% w/v of crystals of about 50 μm to about 600 μm or about 75% w/v of crystals of about 0.5 μm to about 40 μm and about 25% w/v of crystals of about 50 μm to about 600 μm may be provided.

In a preferred form, the present invention may comprise about 25% w/v of crystals of about 0.5 μm to about 40 μm and about 75% w/v of crystals of about 100 μm to about 200 μm. Preferably the composition may comprise about 20% w/v of crystals of about 0.5 μm to about 40 μm and about 80% w/v of crystals of about 100 μm to about 200 μm. Alternatively, about 50% W/V of crystals of about 0.5 μm to about 40 μm and about 50% w/v of crystals of about 100 μm to about 200 μm or about 75% w/v of crystals of about 0.5 μm to about 40 μm and about 25% w/v of crystals of about 100 μm to about 200 μm may be provided.

Crystals of pharmaceutically acceptable salts of the anti-inflammatory steroids of the present invention are also contemplated. The pharmaceutically acceptable salts of the anti-inflammatory steroids described herein are, for example, non-toxic acid addition salts formed with pharmaceutically acceptable acids. Examples include, but are not limited to, hydrochloride, hydrobromide, sulphate and phosphate, acetate, citrate, fumarate, gluconate, lactate, maleate, succinate and tartrate salts. Anti-inflammatory steroids suitable for use in the invention may also include pharmaceutically acceptable metal salts, in particular non-toxic alkali metal salts, with bases. Examples include, but are not limited to, the sodium and potassium salts.

While the present invention is described in the context of the administration of a composition of a single anti-inflammatory steroid which is present in the form of crystals of varying sizes, said crystals being present in a mixture of the ranges of about 0.5 μm to about 40 μm and about 50 μm to about 600 μm or a mixture of the ranges of about 0.5 μm to about 40 μm and about 100 μm to about 200 μm, it should not be understood to be so limited. Combinations of two or more anti-inflammatory steroids, or an anti-inflammatory steroid and another active agent may also be used in the methods of the invention. Combinations of such a nature can be prepared by known methods. Similarly, the invention includes combinations of steroids where the lower size range is a different steroid from the steroid used in the upper size range.

According to a third aspect of this invention, there is provided a method of preparing a pharmaceutically acceptable triamcinolone composition which has an improved therapeutically effective dwell time in the vitreous in a patient, said method comprising the steps of: increasing the concentration of crystals, as compared to crystal composites, in the composition.

According to a fourth aspect of this invention, there is provided a method of preparing a pharmaceutically acceptable triamcinolone composition which has an improved therapeutically effective dwell time in the vitreous in a patient, said method comprising the steps of: increasing the proportion of crystals of the size about 50 μm to about 600 μm in a given triamcinolone preparation compared to the proportion of about 50 μm to about 600 μm crystal composites.

According to a fifth aspect of this invention, there is provided a method of preparing a pharmaceutically acceptable composition which has an improved therapeutically effective dwell time in the vitreous in a patient, said method comprising the steps of: selecting triamcinolone crystals in the size range of about 50 μm to about 600 μm from a triamcinolone composition comprising both crystals and crystal composites. Alternatively, the method may include the steps of: selecting triamcinolone crystals in the size range of about 100 μm to about 200 μm from a triamcinolone composition comprising both crystals and crystal composites. The method may include the additional step of adding said range of crystals to an ophthalmologically acceptable carrier, diluent and/or excipient.

According to a sixth aspect of this invention, there is provided a pharmaceutically acceptable composition prepared according to anyone of the methods described in the third, fourth or fifth aspects of the invention.

The precise formulation used in the pharmaceutical composition of the present invention will vary according to a wide range of commercial and scientific criteria. Preferably, additives to the pharmaceutical composition are suited to the delivery of said pharmaceutical composition as an intravitreal depot injection.

The composition may additionally include at least a pharmaceutically acceptable additive (such as a diluent, carrier, adjunct, excipient or non-toxic, non-therapeutic, non-immunogenic stabilizers and the like). Preferably, the pharmaceutically acceptable additive should be ophthalmologically acceptable, preferably being compatible with the vitreous, and should not leave any vision impairing residue in the eye. Desirably, any pharmaceutically acceptable additive used in the composition may preferably be suited to the delivery of said pharmaceutical composition as an intravitreal depot injection.

Any diluent used in the preparation of the pharmaceutically acceptable composition may preferably be selected so as not to unduly affect the biological activity of the composition. Examples of such diluents which are especially useful for injectable formulations are water, the various saline, organic or inorganic salt solutions, Ringer's solution, dextrose solution, and Hank's solution.

In addition, the pharmaceutical composition may include additives such as other buffers, diluents, carriers, adjuvants or excipients. Any pharmacologically acceptable buffer suitable for application to the eye may be used, e.g., tris or phosphate buffers. Other agents may be employed in the formulation for a variety of purposes. For example, buffering agents, preservatives, co-solvents, surfactants, oils, humectants, emollients, stabilizers or antioxidants may be employed. Water soluble preservatives which may be employed include sodium bisulfite, sodium bisulfate, sodium thiosulfate, benzalkonium chloride, chlorobutanol, thimerosal, phenylmercuric acetate, phenylmercuric nitrate, ethyl alcohol, methylparaben, polyvinyl alcohol, benzyl alcohol and phenylethyl alcohol. These agents may be present in individual amounts of from about 0.001 to about 5% by weight and preferably about 0.01% to about 2%. Suitable water soluble buffering agents that may be employed are sodium carbonate, sodium borate, sodium phosphate, sodium acetate, sodium bicarbonate, etc., as approved by the US FDA for the desired route of administration. These agents may be present in amounts sufficient to maintain a pH of the system of between about 2 to about 9 and preferably about 4 to about 8. As such the buffering agent may be as much as about 5% on a weight to weight basis of the total composition. Electrolytes such as, but not limited to, sodium chloride and potassium chloride may also be included in the formulation.

It has been found that the pharmaceutically acceptable additives or diluents which are provided with some existing products, for example Kenacort® A 40, may include a solvent such as benzyl alcohol, which leads to more rapid dissipation of particles in the vitreous. This finding led to the unexpected finding that the longevity and efficacy of the anti-inflammatory steroid may be diminished by the diluent in which the composition is supplied commercially. The present invention also surprisingly finds that if the particles or crystals are suspended in a normal saline or like solution, the dissolution in the vitreous can be further extended. A balanced salt solution may be used as an alternative to normal saline. A wide variety of balanced salt solutions suitable for the performance of the invention are known to those skilled in the art. For example Ringer's lactate medium may be used. In choosing the balanced salt solution, the immediate efficacy of the active agent can be enhanced compared to Kenacort® A 40. The results relating to choice of diluents are set out in Example 4. The choice of diluent for delivery of the present invention may also be chosen to avoid potentially toxic and/or inflammatory excipients.

Therefore, the present invention is also directed to the diluent in which the anti-inflammatory steroid is suspended. Preferably, the diluent is a balanced salt solution. Most preferably, the balanced salt solution is Ringer's lactate medium.

According to a seventh aspect of this invention, there is provided a method of treating inflammatory eye conditions in a patient requiring said treatment, said method comprising administering to or adjacent to at least an ocular tissue a pharmaceutically acceptable composition as herein disclosed or a pharmaceutically acceptable composition prepared by the method as herein disclosed.

The terms ‘treatment’ or ‘treating’ are used synonymously herein to describe the prevention, slowing, stopping or reversal of the inflammatory eye conditions to which the present invention is directed.

As used herein the phrase “inflammatory eye condition” refers to a disorder or pathological condition of the eye, i.e. ocular disease, which is not normal to the animal in a healthy state that is caused by inflammation or has inflammation as a component to the disease state. Such ocular diseases include, but are not limited to: ocular neovascularization; retinal diseases (such as diabetic retinopathy, sickle cell retinopathy, retinopathy prematurity, macular degeneration (eg early onset macular degeneration, neovascular macular degeneration, age-related macular degeneration)); rubeosis iritis; inflammatory diseases; anterior and posterior uveitis including chronic uveitis; neoplasms (retinoblastoma, pseudoglioma); Fuchs' heterochromic iridocyclitis; neovascular glaucoma; corneal neovascularization (inflammatory, transplantation); sequelae vascular diseases (retinal ischemia, choroidal vascular insufficiency, choroidal thrombosis, carotid artery ischemia); choroidal neovascularization; pterygium; neovascularization of the optic nerve; neovascularization due to penetration of the eye or contusive ocular injury and exudative retinopathies like myopic retinopathies, cystoid macular edema arising from various aetiologies, exudative macular degeneration, diabetic macular edema, central vein occlusion, branch vein occlusion and macular edema arising from laser treatment of the retina.

The individual dosage requirements (i.e., the amount of each dose and the frequency of administration) may vary depending on the severity of the disease, the method of administration, the response of the patient, the patient's health and the patient's medical history. An effective quantity of the compound of interest is preferably employed in the method of the invention. The dosage of compounds used in accordance with the invention varies depending on the compound, formulation of the composition, the method of its administration and the condition being treated. For extraocular and intraocular formulations (delivered by invasive device), the therapeutic composition is delivered at a concentration high enough to achieve a final concentration in the range of about 0.05 mg/ml to about 25 mg/ml within the target ocular compartment (e.g. the posterior chamber for the treatment of retinal diseases).

When administering the steroid by intravitreal injection, the anti-inflammatory steroid should be concentrated to minimise the volume for injection. Preferably when the anti-inflammatory steroid is administered by intraocular delivery, the final concentration of the therapeutic compound is in the range of about 0.05 mg/ml and about 8 mg/ml. More preferably, between about 1 mg/ml and about 7 mg/ml, or between about 1.5 mg/ml and about 6 mg/ml, or between about 2 mg/ml and about 5 mg/ml, or between about 3 mg/ml and about 4 mg/ml. By way of illustration the anti-inflammatory steroid is deposited intravitreally at about 4 mg/ml. This dosage range is subject to the disease condition being treated.

Using a method of the invention, pharmaceutically acceptable compounds may be administered to a patient by any method that leads to delivery of the therapeutic agent to at least the location of the inflammatory eye condition. Preferably, the compositions are administered in unit dosage forms suitable for single administration of precise dosage amounts. Whilst the preferred method of delivery is intra-ocularly, the invention is not limited to intra-ocular delivery. Suitable routes of administration in practicing this invention also include, but are not limited to, topical application, cannular delivery, periorbital injection (including sub-Tenon) into the orbital floor and sub-conjunctival injection, implantation within the eye with or without suturing (for example implantation in the lens capsule), and intravitreal injection. If more than one steroid, or additional active agents, are administered the administration may be by a combination of administration methods, for example delivery of a first steroid by intravitreal injection and a second steroid by topical application.

Administration of the composition is preferably by intraocular injection, although other modes of administration may be effective, if a sufficient amount of the steroid achieves contact with the tissue to be treated. Intraocular injection may be effected by intravitreal injection, aqueous humour injection or injection into the external layers of the eye, such as subconjunctival injection or sub-Tenon injection, or by topical application to the cornea for example as ointment, gel or eye drops, if a penetrating composition comprising the steroid is used. Preferably, the intraocular injection is an intravitreal injection, preferably through self sealing 21-30 gauge needles or other suitably calibrated delivery device. Injection into the eye may be through the pars plana via the self-sealing needle. Preferably a 27 gauge needle may be used for this purpose.

The syringe used in practicing this invention is suitably one which can accommodate a 21 to 30 gauge needle (eg a 23, 24, 25, 26 or 27 gauge needle) and is preferably of a small volume, for example 1.5 mL, or more preferably 0.5 mL. Although it is possible that the needle and syringe may be of the type where the needle is removable from the syringe, it is preferred that the arrangement is of a unitary syringe/needle construction. This would clearly limit the possibility of disengagement of the needle from the syringe. It is also preferred that the arrangement be tamper evident. The compositions of the present invention may therefore be provided in the form of a single unit dose in a pre-prepared syringe, ready for administration.

A suitable style of syringe is, for example, sold under the name of Uniject™ manufactured by Becton Dickinson and Company. In this style of syringe, the material is expelled through the needle into the eye by pressure applied to the sides of a pliable reservoir supplying the needle, rather than by a plunger. As the name implies, the construction of the reservoir and needle forms a single unit.

The frequency of treatment according to the invention is determined according to various factors that include, but are not limited to, the disease being treated, the deliverable concentration of the anti-inflammatory steroid and the method of delivery. Other factors that may affect the frequency of treatment may also include the patient's health and medical history. If delivering the anti-inflammatory steroid by intravitreal injection, the dosage frequency may be monthly or every three months. Preferably, the dosage frequency is less frequent than every three months.

The frequency of dosage may also be determined by observation, with the dosage being delivered when the previously delivered steroid material is visibly degraded, however one should be careful with such a measurement as the steroid material may be visibly degraded, but may exist in dissolved therapeutic levels in the eye. Once a therapeutic result is achieved, the drug can be tapered or discontinued. Occasionally, side effects warrant discontinuation of therapy. In general, an effective amount of the compound is that which provides either subjective relief of symptoms or an objectively identifiable improvement as noted by the clinician or other qualified observer.

Intravitreal injection may be achieved by a variety of methods well known in the art. For example, the eye may be washed with a sterilising agent such as Betadine® and the steroid injected in an appropriate carrier with a fine gauge needle (eg 27 gauge) at a position in the eye such that the steroid crystals will settle to the posterior pole towards the ventral surface. It may be necessary to prepare the eye for injection by application of positive pressure prior to injection. In some cases, paracentesis may be necessary. Local anaesthetic or general anaesthetic may be necessary.

The invention also provides a pharmaceutically acceptable composition of an anti-inflammatory steroid or pharmaceutically acceptable salt thereof which is present in the form of crystals of varying sizes, said crystals being present in a mixture of the ranges of the size ranges as herein described in a biocompatible, biodegradable matrix, for example in a topical form.

Topical application of the anti-inflammatory steroid or pharmaceutically acceptable salt thereof may be as an in situ gellable aqueous composition. Such a composition comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye. Indeed, it can be advantageous to formulate a composition of the invention as a gel, to minimize loss of the composition immediately upon administration, as a result, for example, of lacrimation caused by reflex blinking. Although it is preferred that such a composition exhibit further increase in viscosity or gel stiffness upon administration, this is not absolutely required if the initial gel is sufficiently resistant to dissipation by lacrimal drainage to provide the effective residence time specified herein.

To prepare a topical formulation for the treatment of ophthalmological disorders, a therapeutically effective amount of the anti-inflammatory steroid or pharmaceutically acceptable salt thereof is placed in an ophthalmological vehicle as is known in the art. The amount of the therapeutic compound to be administered and the concentration of the compound in the topical formulations depend upon the diluent, delivery system or device selected, the clinical condition of the patient, the side effects and the stability of the compound in the formulation. Thus, the physician employs the appropriate preparation containing the appropriate concentration of the therapeutic compound and selects the amount of formulation administered, depending upon clinical experience with the patient in question or with similar patients.

The method of the present invention may be performed alone, or in combination with one or more other therapies such as photodynamic therapy, laser treatment, or one or more biological or pharmaceutical treatments.

Where laser treatment of the retina is indicated, administration of an anti-inflammatory steroid may be carried out by injection before or after the laser treatment.

Other substances, such as antibiotics and anti-angiogenesis agents may be injected with the anti-inflammatory steroid in combined therapies. Two such anti-angiogenic agents designed to block the actions of VEGF on endothelial cells that can be employed in the method of the invention are: (a) Lucentis® made by Genentech; and (b) Macugen® made by Eyetech Pharmaceuticals. Lucentis® and Macugen® are compounds that are injected into the vitreous and are potent anti-angiogenic compounds. In a highly preferred form, the pharmaceutical composition of the invention will comprise an anti-inflammatory steroid as described and an anti-angiogenic agent such as Lucentis® or Macugen®.

Lucentis® (ranibizumab), formerly known as rhuFab V2 or AMD-Fab is a humanized, therapeutic anti-VEGF (vascular endothelial growth factor) antibody fragment developed at Genentech to bind and inhibit VEGF, a protein that plays a critical role in angiogenesis (the formation of new blood vessels). Lucentis is designed to block new blood vessel growth and reduce leakage, which are thought to lead to wet AMD disease progression. When administered in conjunction with pharmaceutical compositions prepared according to the present invention Lucentis should be administered in either about 300 or about 500 microgram doses for four doses.

Macugen® (pegaptanib sodium, anti-VEGF apatamer or EYE001) made by Eyetech Pharmaceuticals, consists of a synthetic fragment of genetic material that specifically binds to the VEGF molecule and blocks it from stimulating the receptor on the surface of endothelial cells. When administered in conjunction with pharmaceutical compositions prepared according to the present invention Macugen® should be administered in a dose ranging from either about 0.3 mg to about 3.0 mg every four or six weeks.

In another aspect of the invention the anti-inflammatory steroid is prepared in combination with a glucocorticoid (e.g. prednisolone, prednisone), an oestrogen (e.g. oestrodiol), an androgen (e.g. testosterone) retinoic acid derivatives (e.g. 9-cis-retinoic acid, 13-trans-retinoic acid, all-trans retinoic acid), a vitamin D derivative (e.g. calcipotriol, calcipotriene), a non-steroidal anti-inflammatory agent, a vitamin D derivative, an anti-infective agent, a protein kinase C inhibitor, a MAP kinase inhibitor, an anti-apoptotic agent, a growth factor, a nutrient vitamin, an unsaturated fatty acid, and/or ocular anti-infective agents, for the treatment of the ophthalmic disorders set forth herein. In still other embodiments of the invention, a mixture of these agents may be used.

Ocular anti-infective agents as described herein include, but are not limited to, penicillins (ampicillin, aziocillin, carbenicillin, dicloxacillin, methicillin, nafcillin, oxacillin, penicillin G, piperacillin, and ticarcillin), cephalosporins (cefamandole, cefazolin, cefotaxime, cefsulodin, ceftazidime, ceftriaxone, cephalothin, and moxalactam), aminoglycosides (amikacin, gentamicin, netilmicin, tobramycin, and neomycin), miscellaneous agents such as aztreonam, bacitracin, ciprofloxacin, clindamycin, chloramphenicol, cotrimoxazole, fusidic acid, imipenem, metronidazole, teicoplanin, and vancomycin), antifungals (amphotericin B, clotrimazole, econazole, fluconazole, flucytosine, itraconazole, ketoconazole, miconazole, natamycin, oxiconazole, and terconazole), antivirals (acyclovir, ethyldeoxyuridine, foscamet, ganciclovir, idoxuridine, trifluridine, vidarabine, and (S)-1-(3-dydroxy-2-phospho-nyluethoxypropyl) cytosine (HPMPC)), antineoplastic agents (cell cycle (phase) nonspecific agents such as alkylating agents (chlorambucil, cyclophosphamide, mechlorethamine, melphalan, and busulfan), anthracycline antibiotics (doxorubicin, daunomycin, and dactinomycin), cisplatin, and nitrosoureas), antimetabolites such as antipyrimidines (cytarabine, fluorouracil and azacytidine), antifolates (methotrexate), antipurines (mercaptopurine and thioguanine), bleomycin, vinca alkaloids (vincrisine and vinblastine), podophylotoxins (etoposide (VP-16)), and nitrosoureas (carmustine, (BCNU)), immunosuppressant agents such as cyclosporin A and SK506, and anti-inflammatory or suppressive factors (inhibitors), and inhibitors of proteolytic enzymes such as plasminogen activator inhibitors. Doses for topical and sub-conjunctival administration of the above agents, as well as intravitreal dose and vitreous half-life may be found in Intravitreal Surgery Principles and Practice, Peyman G A and Shulman, J Eds., 2nd edition, 1994, Appleton-Longe, the relevant sections of which are expressly incorporated by reference herein.

### EXAMPLES

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

### Example 1

Preparation of Composition 1

A sample of TA in the form of Kenacort® A 40 was mixed in equal proportions with a sample of TA from Tianjin Tianyao Pharmaceuticals, Tianjin, China (the “Chinese sample”). This achieves a composition containing TA with particle sizes ranging from 1 μm to 100 μm.

Preparation of Composition 2

A sample of TA in the form of Kenacort® A 40 is fractionated to extract crystals of a size range between about 1 μm to 20 μm. Further, the Chinese sample of TA is fractioned to extract crystals of a size range between about 80 μm and 120 μm. The fractioned material is then mixed in a ratio of 4 to 1 w/v. This achieves a composition containing TA with particle sizes ranging from 1 μm to 120 μm.

Preparation of Composition 3

A sample of TA in the form of Kenacort® A 40 is fractionated to extract crystals of a size range between about 5 μm to 20 μm. Further, the Chinese sample of TA is fractioned to extract crystals of a size range between about 105 μm and 120 μm. The fractioned material is then mixed in a ratio of 1 to 1 w/v. This achieves a composition containing TA with particle sizes ranging from 1 μm to 120 μm.

Preparation of Composition 4

A sample of TA in the form of Kenacort® A 40 is fractionated to extract crystals of a size range between about 5 μm to 15 μm. Further, the Chinese sample of TA is fractioned to extract crystals of a size range between about 110 μm and 120 μm. The fractioned material is then mixed in a ratio of 1 to 4 w/v. This achieves a composition containing TA with particle sizes ranging from 1 μm to 120 μm.

### Example 2

Particle Size Analysis

Particle size analysis on new and aged Kenacort® A 40 samples using a Malvern Laser Scattering method showed the TA particles to have a mean size of 13 μm. The distribution was very narrow in both cases.

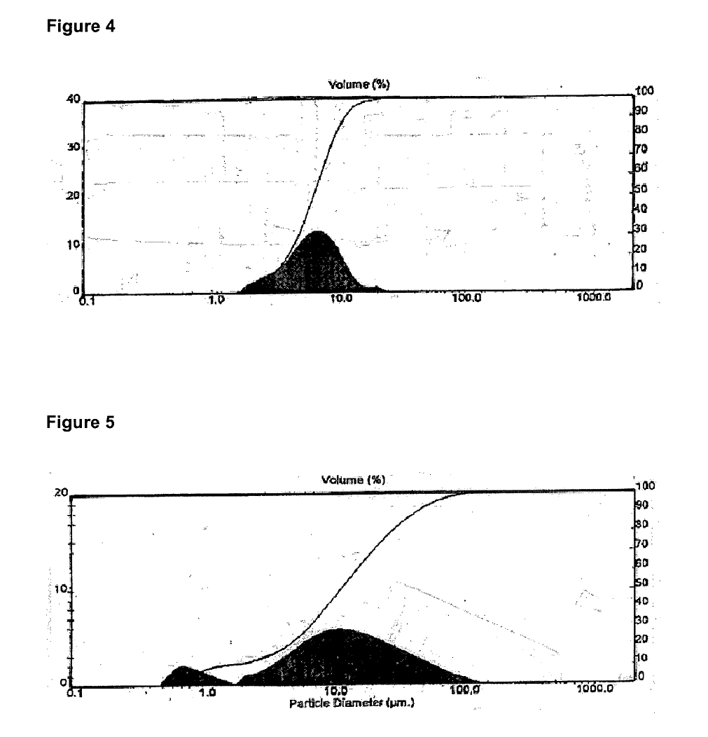

Analysis of material from SICOR S.p.A, Milan, Italy (the “Italian sample”) is presented in FIG. 4.

Analysis of a sample of TA from the Chinese sample which was reported to be micronised, showed a mean particle size of 11.48 μm (FIG. 5) which was similar to the Kenacort® A 40 material. However, the distribution of particle sizes was wider than for the Kenacort® A 40 samples.

One significant finding of these results is that the TA crystals do not appear to grow in aged samples and the distribution of particles in the Kenacort® A 40 product is well controlled within a fairly narrow range. The wider distribution of particles sizes in the Chinese sample would be expected to provide a more constant rate of release over a longer period of time than the Kenacort® A 40 product.

### Example 3

Dissolution Studies of Triamcinolone Acetonide—Comparative Studies

Method

Samples were prepared by dispensing 10 mg of sample into 10 mL of a balanced salt solution comprising 1% Tween 80. The sample in solution was then subjected to vigorous shaking and a number of rapid expulsions through a 22 gauge needle. 2 mL of this suspension was then filtered through a 22 μm Durapore membrane filter which retained essentially all of the particles. The filter is then rinsed with 2 mL water.

This method of sample preparation avoids the problem of particles formed of large agglomerations of crystals which appear to dissolve as large crystals and therefore bias results.

Dissolution was performed using a flow through system that pumps a degassed solvent solution of 20% methanol in water through the dissolution chamber at around 11 mL per minute. This flow rate maintained sink conditions wherein a large surplus of solvent/absorbant/carrier capacity is maintained throughout the experiment. The combination of flow rate and solvent solution dissolves the TA crystals at a rate that enables a flow-through cell to record absorbance values in the acceptable range for virtually all of the 30 minute run time and for the experiment to be completed in a reasonable period.

The solubility of TA in the solvent solution is approximately 2-3 times that of TA in water alone. However, the wetting characteristics of the solvent solution are superior to those of water alone.

Approximately 2 mg of sample was prepared on a filter as outlined above. The filter was then placed in a modified stainless steel swinnex adaptor that was used as the dissolution chamber. The effluent from the dissolution chamber was passed through a low volume flow-through cell in a Hitachi 1001 spectrophotometer with the wavelength set at 238 nm. The absorbance was recorded on a chart recorder with 2 absorbance units causing 100% pen deflection.

Dissolution studies were carried out for a period of 30 minutes with a flow rate of 11 mL per minute. Average absorbance values over 2 minute periods were calculated. The experiment was done in duplicate and the two absorbance values for each sample period were averaged.

Results

Chinese Sample

TABLE 1
Chinese Sample

| Period | Absorbances of A | | Average | % | Cumulative % |
|---|---|---|---|---|---|
| (minutes) | (absorbance units) | | Absorbance | Dissolved | Dissolved |
| 1 | 1.2 | 1.2 | 1.2 | 53.9 | 53.9 |
| 2 | 0.395 | 0.360 | 0.378 | 17.0 | 70.9 |
| 3 | 0.195 | 0.195 | 0.195 | 8.8 | 79.7 |
| 4 | 0.125 | 0.126 | 0.126 | 5.7 | 85.4 |
| 5 | 0.082 | 0.080 | 0.081 | 3.6 | 89.0 |
| 6 | 0.060 | 0.058 | 0.059 | 2.7 | 91.7 |
| 7 | 0.045 | 0.040 | 0.042 | 1.9 | 93.6 |
| 8 | 0.036 | 0.030 | 0.033 | 1.5 | 95.1 |
| 9 | 0.030 | 0.024 | 0.027 | 1.2 | 96.3 |
| 10 | 0.025 | 0.020 | 0.022 | 1.0 | 97.3 |
| 11 | 0.020 | 0.016 | 0.018 | 0.8 | 98.1 |
| 12 | 0.015 | 0.014 | 0.014 | 0.7 | 98.8 |
| 13 | 0.010 | 0.012 | 0.011 | 0.5 | 99.3 |
| 14 | 0.008 | 0.010 | 0.009 | 0.4 | 99.7 |
| 15 | 0.006 | 0.008 | 0.007 | 0.3 | 100 |
| | 2.252 | 2.193 | 2.222 | | |

Reviewing the results in Table 1, the sum of the absorbance values is 2.222 (average 0.148) and therefore, by taking into account the volume of dissolution fluid passed through the chamber, one can calculate the amount of TA dissolved over this period (using an E1% of 350) as being 1.40 mg.

This is substantially less than the 2 mg expected, based on the amount of sample applied to the dissolution chamber.

However, the sample solution was prepared several hours before being added to the dissolution chamber, which may have resulted in a significant amount of material dissolving prior to commencement of the experiment. This would have resulted in a reduced initial dissolution rate, but would not have affected the dissolution rate at later times.

Kenacort® A 40 Sample

TABLE 2
Kenacort ® A 40

| Period | Absorbances of A | | Average | % | Cumulative |
|---|---|---|---|---|---|
| (Minutes) | (absorbance units) | | Absorbance | Dissolved | % Dissolved |
| 1 | 1.21 | 1.21 | 1.21 | 42.3 | 42.3 |
| 2 | 0.660 | 0.600 | 0.630 | 22.0 | 64.3 |
| 3 | 0.390 | 0.345 | 0.368 | 12.8 | 77.1 |
| 4 | 0.235 | 0.210 | 0.223 | 7.8 | 84.9 |
| 5 | 0.140 | 0.130 | 0.135 | 4.7 | 89.6 |
| 6 | 0.090 | 0.086 | 0.088 | 3.1 | 92.7 |
| 7 | 0.060 | 0.060 | 0.060 | 2.1 | 94.8 |
| 8 | 0.040 | 0.040 | 0.040 | 1.4 | 96.2 |
| 9 | 0.030 | 0.030 | 0.030 | 1.0 | 97.2 |
| 10 | 0.022 | 0.022 | 0.023 | 0.8 | 98.0 |
| 11 | 0.018 | 0.018 | 0.018 | 0.6 | 98.6 |
| 12 | 0.015 | 0.015 | 0.015 | 0.5 | 99.1 |
| 13 | 0.010 | 0.010 | 0.010 | 0.4 | 99.5 |
| 14 | 0.008 | 0.008 | 0.008 | 0.3 | 99.8 |
| 15 | 0.006 | 0.006 | 0.006 | 0.2 | 100 |
| | 2.934 | 2.790 | 2.862 | | |

Reviewing the results in Table 2, the sum of the absorbance values is 2.862 (average 0.191) and therefore, by taking into account the volume of dissolution fluid passed through the chamber, one can calculate the amount of TA dissolved over this period (using an E1% of 350) as being 1.80 mg.

This is substantially less than the 2 mg expected, based on the amount of sample applied to the dissolution chamber.

However, the sample solution was prepared from a suspension of 40 mg/ml. It is probable that a greater quantity than 40 mg was transferred from the ampoule (due to overage) however some would no doubt be in solution and again over time the particles would dissolve. This sample was used reasonably quickly after preparation.

Italian Sample

TABLE 3
Italian Sample

| Period | Absorbances of A | | Average | % | Cumulative % |
|---|---|---|---|---|---|
| (minutes) | (absorbance units) | | Absorbance | Dissolved | Dissolved |
| 1 | 1.5 | 1.5 | 1.5 | 49.2 | 49.2 |
| 2 | 0.900 | 0.910 | 0.905 | 29.7 | 78.9 |
| 3 | 0.410 | 0.420 | 0.415 | 13.6 | 92.5 |
| 4 | 0.160 | 0.150 | 0.155 | 5.1 | 97.6 |
| 5 | 0.054 | 0.050 | 0.052 | 1.7 | 99.3 |
| 6 | 0.020 | 0.016 | 0.018 | 0.6 | 99.9 |
| 7 | 0.004 | 0.004 | 0.004 | 0.1 | 100 |
| 8 | 0.000 | 0.000 | 0.000 | 0.0 | 100 |
| 9 | 0.000 | 0.000 | 0.000 | 0.0 | 100 |
| 10 | 0.000 | 0.000 | 0.000 | 0.0 | 100 |
| 11 | 0.000 | 0.000 | 0.000 | 0.0 | 100 |
| 12 | 0.000 | 0.000 | 0.000 | 0.0 | 100 |
| 13 | 0.000 | 0.000 | 0.000 | 0.0 | 100 |
| 14 | 0.000 | 0.000 | 0.000 | 0.0 | 100 |
| 15 | 0.000 | 0.000 | 0.000 | 0.0 | 100 |
| | 3.048 | 3.050 | 3.049 | | |

Reviewing the results in Table 3, the sum of the absorbance values is 3.049 (average 0.203) and therefore, by taking into account the volume of dissolution fluid passed through the chamber, one can calculate the amount of TA dissolved over this period (using an E1% of 350) as being 1.92 mg.

Findings

Over a ten minute period both the Kenacort® A 40 and the Chinese sample dissolved to a similar extent (89.7% and 89.0% respectively) whereas 99.3% of the Italian sample had dissolved. The Italian sample had completely dissolved by 14 minutes whereas the Kenacort® A 40 and the Chinese sample were only 98% and 97.3% dissolved after 20 minutes.

There appears to be little difference in the dissolution characteristics for the Kenacort® A 40 and the Chinese sample.

### Example 4

Equilibrium Solubility of Triamcinolone Acetonide in Various Media

An HPLC assay method was developed for TA. This was used to determine the equilibrium solubility for TA in various media. It was considered that a solution of acetonitrile may be advantageous for the maintenance of sink conditions, wherein a large surplus of solvent/absorbant/carrier capacity is maintained. The concentration of TA in the supernatant of Kenacort® A 40 was also investigated. Results are presented in Table 4.

TABLE 4

| Media | Triamcinolone acetonide conc. (mg/L) |
|---|---|
| Kenacort ® A 40 | 24.6 |
| Distilled water | 8.8 |
| Normal saline | 8.2 |
| 5% Acetonide | 5.2 |
| 10% Acetonide | 9.1 |
| 30% Acetonide | 128.0 |

It is noteworthy that the concentration of TA in the supernatant of Kenacort® A 40 is considerably higher than that in water or saline solution. The equilibrium concentration of TA increases with increasing concentrations of acetonitrile. This may explain the initial clinical effect observed with the supernatant alone. The high concentration of TA may be due to the presence of benzyl alcohol, or to micellar solubilisation in the surfactant employed in the Kenacort® A 40 formulation.

### Example 5

Dissolution Studies

Initial dissolution studies were performed on the Chinese sample using various concentrations of acetonitrile in water as the solvent. Results are set out in Table 5.

TABLE 5

| Triamcinolone | | |
|---|---|---|
| Acetonitrile Conc (w/v) | Time (min) | mg Dissolved |
| 30% | 10 | 48 |
| 20% | 10 | 24.20 |
| | 30 | 36.08 |
| | 60 | 41.73 |
| | 120 | 44.87 |
| | 240 | 46.19 |
| 15% | 15 | 23.83 |
| | 30 | 29.85 |
| | 60 | 36.44 |
| | 120 | 40.42 |
| | 240 | 43.25 |
| 12.5% | 30 | 24.63 |
| | 60 | 28.84 |
| | 120 | 32.03 |
| | 240 | 34.71 |
| 5.0% | 30 | 5.19 |
| | 60 | 7.37 |
| | 120 | 10.44 |
| | 180 | 11.35 |
| 0.0% | 30 | 2.26 |
| | 60 | 6.13 |
| | 120 | 8.78 |
| | 180 | 11.44 |

It is clear that as the concentration of acetonitrile increases, the dissolution rate of the TA in the sample increases.

### Example 6

Fractionation of Triamcinolone Acetonide Samples—Density Centrifugation

Dry powder samples of TA were fractionated by density centrifugation in distilled water at 4° C.

### Example 7

Fractionation of Triamcinolone Acetonide Samples—Filtration

Samples of TA suspended in distilled water were fractionated by passage through a series of Millipore filters of maximum pore size 10 μm and 50 μm.

### Example 8

Structure of Triamcinolone Acetonide Crystals from Various Sources

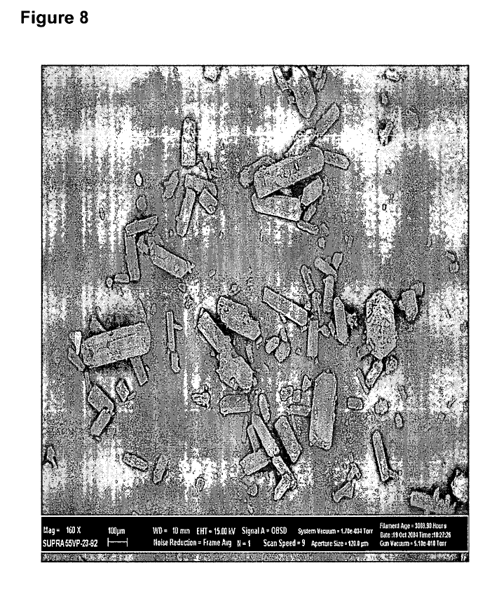

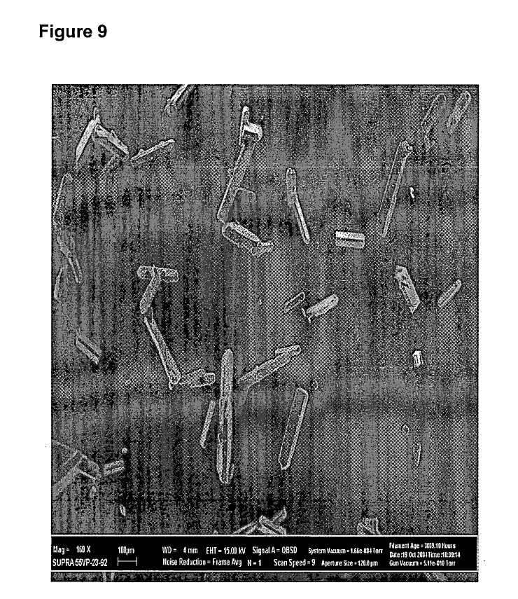

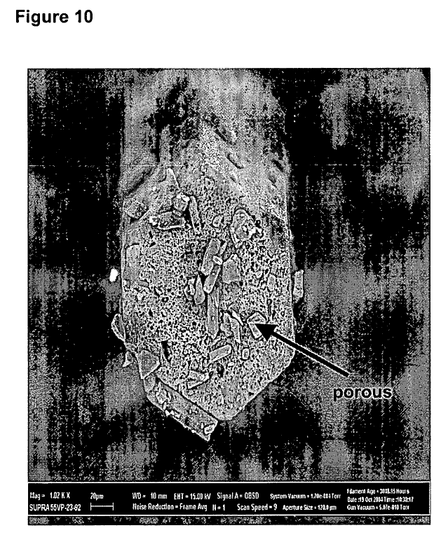

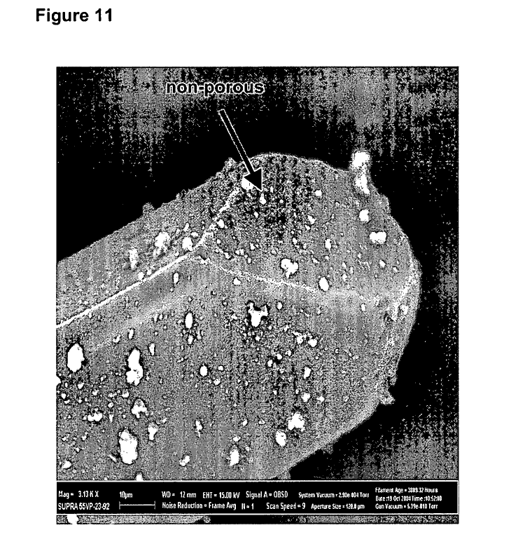

Crystals of TA from Farmabios S.p.A, Gropello Cairoli, Italy (“Farmabios”) and NewChem S.p.A, Milan, Italy (“NewChem”) were viewed by scanning electron microscopy (SEM). As can be seen in FIGS. 8 and 9, crystals from Farmabios appeared to be more “chunky” whilst those from NewChem were more “needle-like”, having a higher aspect ratio. Furthermore, differences in the porosity of the crystals are observable in FIGS. 10 and 11.

### Example 9

Particle Size Distributions of Triamcinolone Acetonide Crystals from Various Sources

Method

Samples of micronised Farmabios and non-micronised Farmabios and NewChem TA were analysed for their particle size distribution by laser light scattering. Particle size distributions were determined on a Malvern Mastersizer S, using the MS7 magnetically stirred cell at room temperature, and the 3NDD presentation. Background measurements for MilliQ water were obtained. Samples were prepared as a concentrated suspension of 5 mg in 250 μL of suspending solution (0.25% Tween 20, 0.25% sodium carboxymethylcellulose solution [NaCMC] in MilliQ water in an Eppendorf tube) and the entire contents of the suspension added to the magnetically stirred cell containing ˜20 mL of MilliQ water under half maximum stirring. This speed was previously optimised for dispersion of large particles of TA. If required, the suspension was diluted further with water to obtain obscuration figures of 15-20%. Measurements were conducted over 2500 scans.

Results

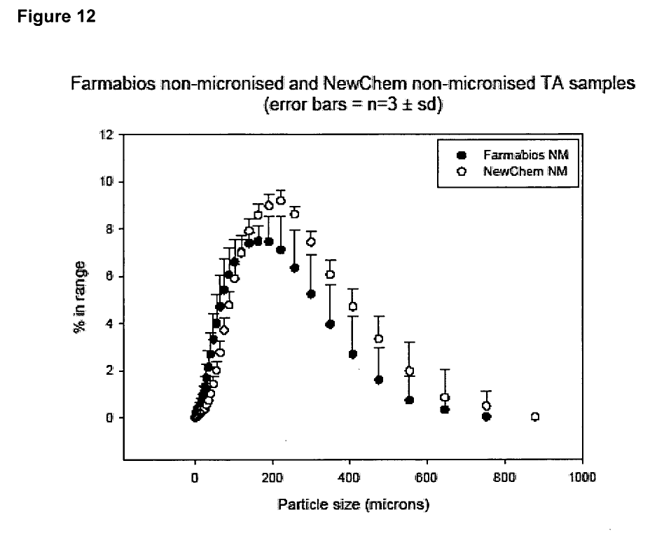

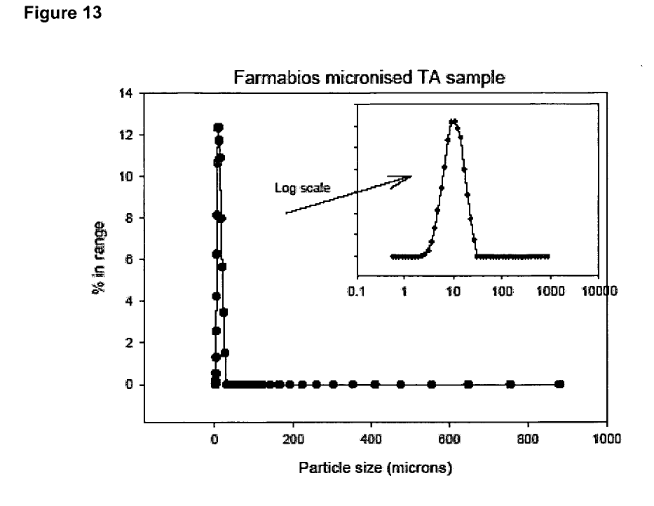

FIGS. 12 and 13 shows the distribution of particle sizes obtained for the three TA samples. The D(v,0.9) values obtained for the three samples are tabulated below in Table 6; the D(v,0.9) is best understood to be the 90% median value, i.e. only 10% of the particles are estimated to be a size greater than this value.

TABLE 6

| Material | D(v, 0.9) μm |
|---|---|
| Farmabios (non-micronised) | 289 |
| NewChem (non-micronised) | 324 |
| Farmabios (micronised) | 17 |

The D(v, 0.9) values for the two non-micronised samples were similar. The micronised material was larger than expected, indicative of agglomerate formation (no sonication was applied to the samples).

### Example 10

Dissolution of Triamcinolone Acetonide Samples From Different Sources

Method

Dissolution Studies

A 20 mg/mL suspension of TA was prepared in suspending solution (0.25% Tween 20, 0.25% NaCMC solution in MilliQ water). 400 mL of release medium (saline, 0.9% NaCl in MilliQ water) was placed into an Erweka 1 L dissolution vessel, which was placed into the Erweka USP2 Dissolution Apparatus thermostatted to 37° C. When the release medium had equilibrated under stirring at 100 rpm to 37° C., the suspension of TA was mixed by vortexing and 100 μL (2 mg) of the suspension immediately pipetted into the release medium at time=0.

At intervals thereafter determined by the system under study, 1 mL of release medium was drawn into a 1 mL disposable syringe. A syringe filter (Acrodisk 0.2 μm Supor, Pall Gelman) was then attached, and 0.8 mL of the sample expelled from the syringe through the filter and back into the release medium.

The final ˜200 μL was collected into an Eppendorf tube, and 100 μL accurately pipetted into a second Eppendorf tube containing 100 μL of acetonitrile, and mixed by vortexing. This mixture was then transferred to an HPLC vial glass insert for injection.

The process was repeated at each time point, using a new syringe and syringe filter.

Filter Validation

Solutions at 1% and 10% of saturation were prepared from a stock of saline saturated with TA (15 μg/mL). The solutions were filtered through a syringe filter and aliquots (200 μL) of the filtrate were collected. HPLC showed that the concentration of TA in the filtrate had reached 94% and 95% respectively after 600 μL of TA solution had passed through the filter. Consequently, an 800 μL prefiltration volume (see dissolution method above) was assumed sufficient to ensure that the filtrate concentration was representative of the dissolved TA concentration in the dissolution vessel.

HPLC Method

A mobile phase consisting of 40% acetonitrile and 60% MilliQ water was prepared and 0.1% trifluoroacetic acid added before filtration. A Beckman Gold system and Waters 717 Autosampler, equipped with a 168 PDA detector was used, and the mobile phase was pumped through a Beckman Ultrasphere column C8 column (4.6 mm×25 cm, 5 μm) at 1.2 mL/min. The retention time of TA was approximately 7 mins, after 100 μL injection.

Standards were prepared in mobile phase at 1000, 500, 250, 100, 75 and 50 ng/mL from a stock solution of TA in acetonitrile at 1 mg/mL. The 50 ng/mL standard was just inside acceptable precision limits (% CV=20%, n=4 injections), while the other concentrations gave % CV<10% in all cases, with linearity >0.999. Aqueous samples were diluted 50% v/v in acetonitrile prior to injection to ensure miscibility with mobile phase.

Results

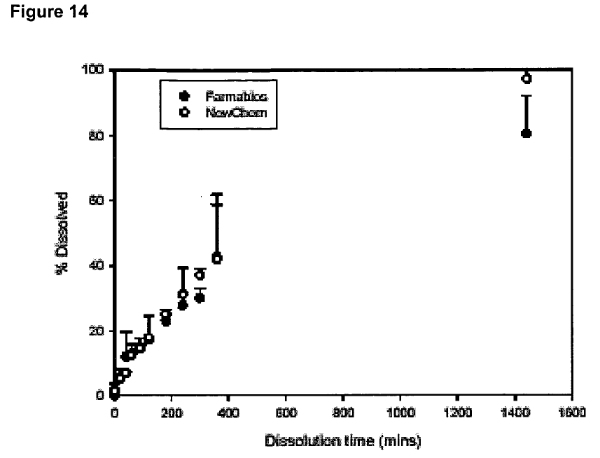

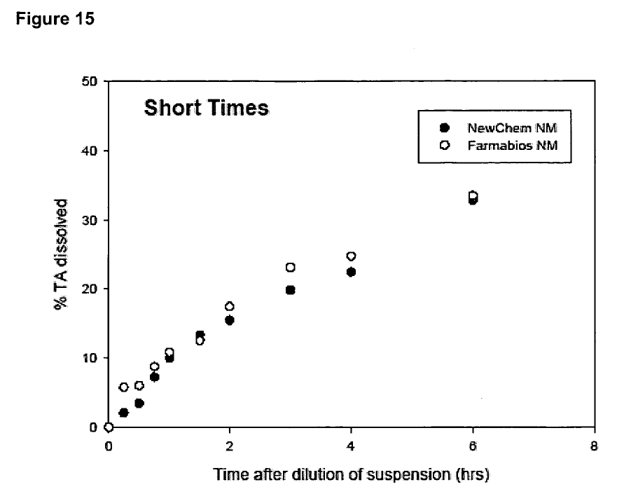

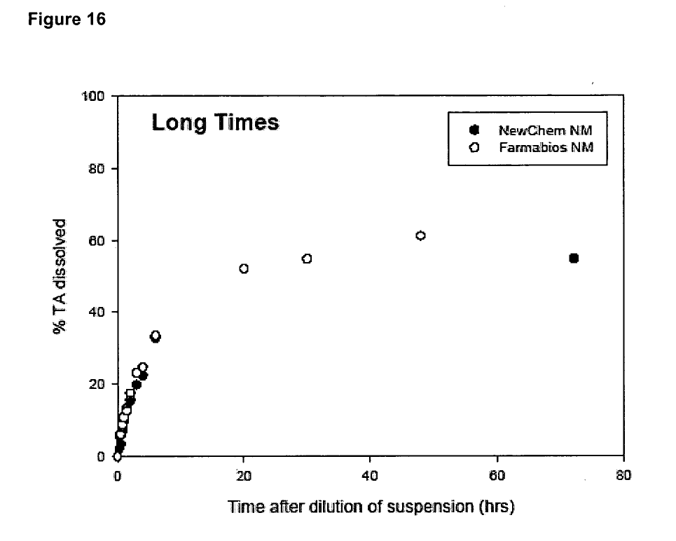

It was found that the non-micronised TA sourced from NewChem behaved in an identical fashion to the non-micronised TA sourced from Farmabios. Despite the differences in crystal shapes and porosity between TA sourced from either Farmabios or NewChem, the preliminary data in FIG. 14 and further data in FIGS. 15 and 16 show that there is no discernable difference in dissolution rate between the non-micronised TA from either source under the conditions used in these studies.

Note that the gap between sampling points in FIG. 16 for the NewChem sample is due to the dissolution being left to run over the weekend (no sampling) whereas the Farmabios sample was run during the week, and intermediate samples were taken in that case.

The reason for the plateau in dissolution at around 60% in FIG. 16 may be attributed to greater difficulty in transferring all of the non-micronised stock sample of TA in suspending solution from the Eppendorf in which it was prepared, compared to the micronised suspension. More non-micronised material may be left in the pipette tip used to transfer the TA suspension into the dissolution vessel.

### Example 11

Dissolution of Micronised and Non-Micronised Triamcinolone Acetonide

Methods

Dissolution studies were carried out on micronised and non-micronised Farmabios TA samples and mixtures of 80:20, 50:50 and 20:80 w/w micronised:non-micronised TA. The basic method for measuring dissolution used was similar to that described above in Example 10. The two individual samples and the three mixtures were prepared by weight (2.0 mg) in an Eppendorf tube and 1 mL of suspending medium (0.25% Tween 20 and 0.25% sodium carboxymethylcellulose) added to wet the powders immediately prior to addition of the contents to a dissolution bath (Erweka) containing 400 mL of saline (0.9% NaCl in MilliQ water) at 37° C. The dissolution medium was stirred with a USP2 compliant paddle at 100 rpm. Samples were withdrawn from the dissolution bath at set time intervals by syringe and filtered, with the final 100 μl of filtrate diluted with 100 μl of acetonitrile prior to injection onto an HPLC to determine the TA concentration in the dissolution medium.

Results

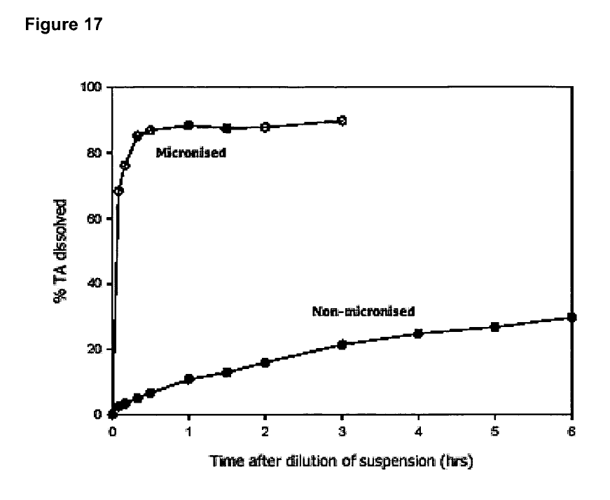

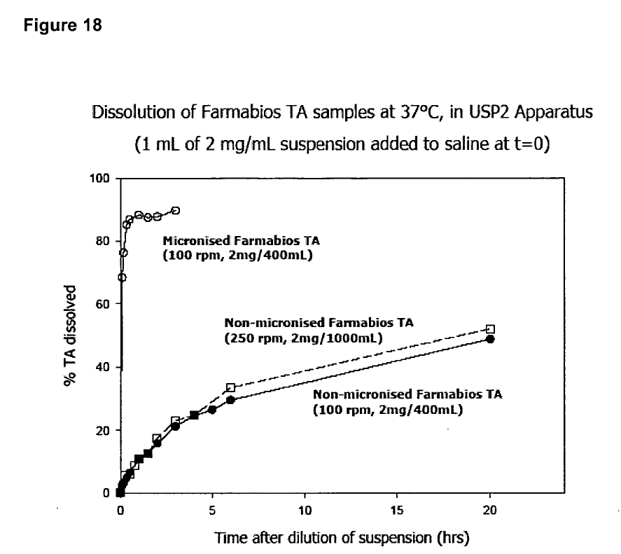

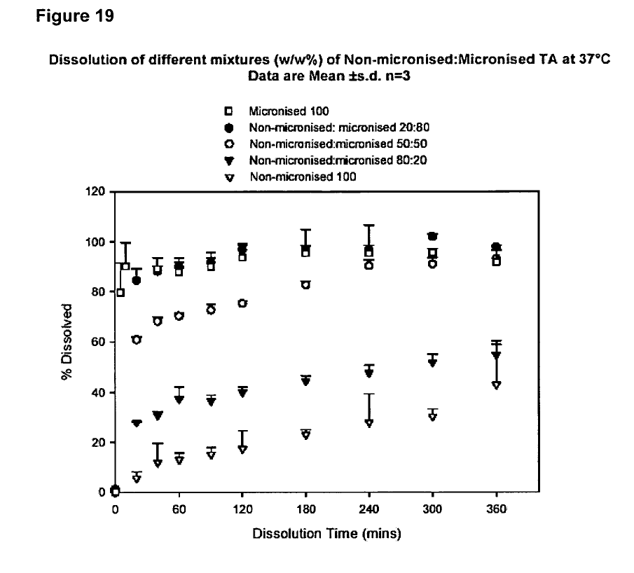

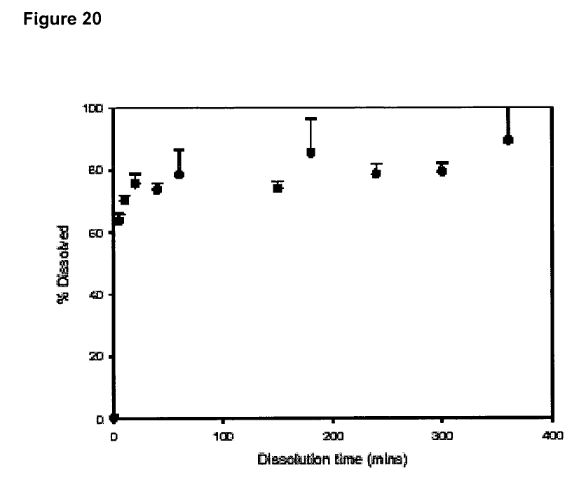

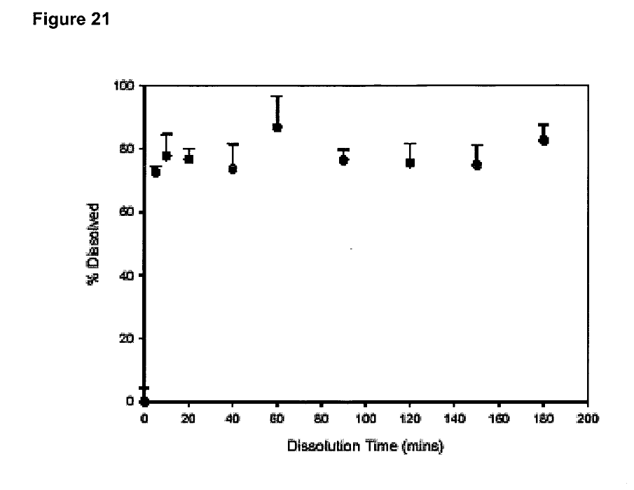

The dissolution profiles for micronised and non-micronised TA from Farmabios over 6 hrs (FIG. 17) and 20 hrs (FIG. 18) and mixtures of micronised and non-micronised TA in FIG. 19 show a steady progression to slower dissolution rates with increasing non-micronised TA content. In FIG. 20, the 100% micronised TA follows the same trend.

### Example 12

Dissolution Studies in Viscous Media

Method

Dissolution studies were carried out in a viscous gel prepared by addition of 3% sodium carboxymethyl cellulose (CMC) medium viscosity grade, to saline. This provided dissolution data in a system more closely resembling the in vivo situation, but still retaining a sink condition. The basic method for measuring dissolution used was that described above in Example 10. However, samples, (0.5 mL) were centrifuged for three minutes after removal from the gel prior to determination of TA content. The centrifugation step was necessary as the gel was not readily filterable.

Results

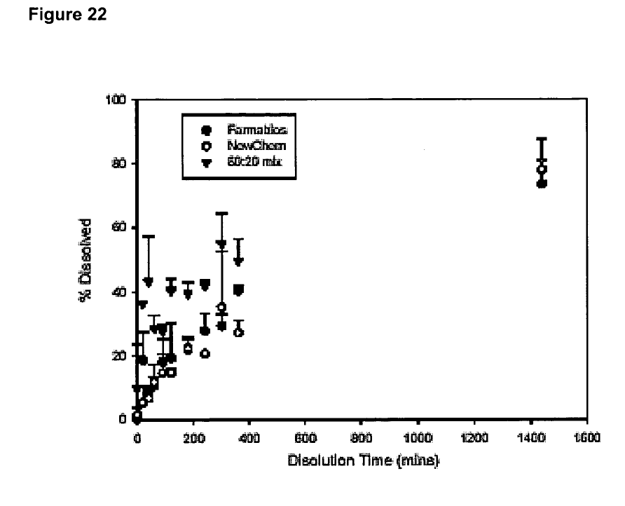

Despite some variability in results, the data illustrated in FIGS. 21 and 22 essentially shows the same trend as observed for micronised and non-micronised TA and mixtures dissolved in saline, as shown in Example 11 above.

### Example 13

Simulated Eve Diffusion Apparatus

In order to illustrate the dissolution of TA in a viscous medium close to the clinical situation, an in vitro dissolution cell was utilised as the basis for a simulated eyeball.

Method

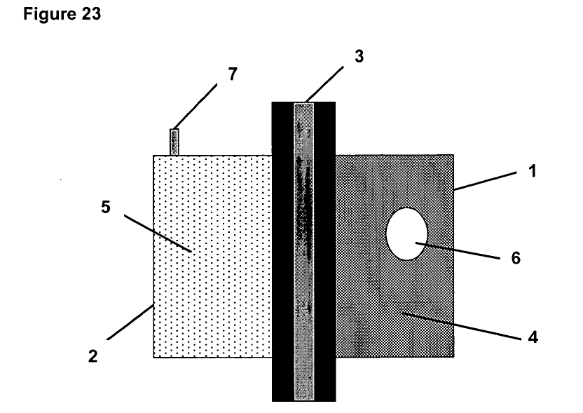

The cell (FIG. 23) consists of two compartments (donor chamber (1) and receptor chamber (2)), each approximately 9 mL in volume, separated by a dialysis membrane (3) (Spectropor 3, 3500 MWCO). Gel (4) (1% hyaluronic acid (HA) in saline; simulating vitreous humour) was placed in the donor chamber (1) and saline release medium (5) placed in the receptor chamber (2) on the other side of the membrane (3). The membrane (3) allows passage of TA but not HA, ensuring the gel (4) remains intact. A composition comprising TA (6) was then injected into the gel (4) in the donor chamber (1) and the appearance of free drug in the saline release medium (5) in the receptor chamber (2) was monitored by HPLC by removing the entire contents of the receptor chamber (2) through sampling port (7). The saline release medium (5) in the receptor chamber (2) was replaced each time a sample was taken. The entire apparatus was immersed in a water bath at 37° C. with shaking (100 rpm) throughout the whole experiment.

Four mg of Farmabios TA in either the micronised or non-micronised form was injected into the gel as a 40 mg/mL suspension. The recovered samples from the receptor chamber were diluted up to 10 ml with saline, and HPLC conducted on the samples to determine the amount of TA.

Results

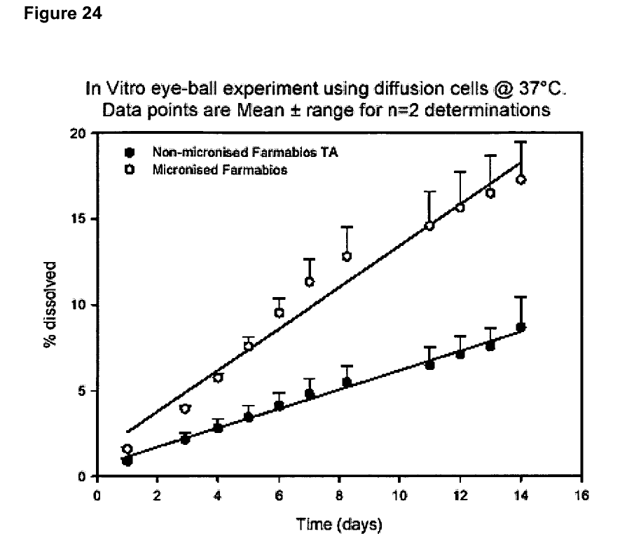

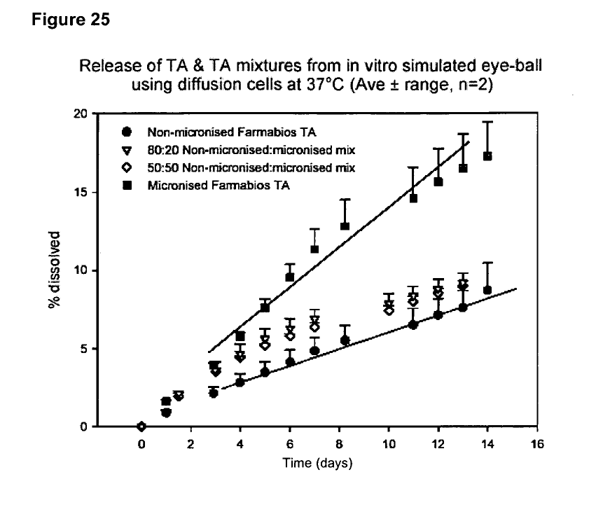

As shown in FIGS. 24 and 25, the cumulative release profiles for the micronised or non-micronised formulations over the two weeks are different, with the micronised material appearing in the receptor solution more rapidly than the non-micronised TA.

### Example 14

Fractionation of Triamcinolone Acetonide Samples—Sedimentation

Method

A concentrated suspension of TA (1 mL, 100 mg/mL) was added by pipette to the top of a glass tube containing 400 mL of MilliQ water, (1000 mm high×25 mm internal diameter) fitted with a clamp at the base. The particles were permitted to settle under gravity for 70 seconds (sufficient time for most of the larger particles to visibly separate into a different ‘zone’ to the smaller particles), upon which the clamp was released and 150 mL of the dispersion collected to separate the major ‘zones’. The suspensions were then filtered separately through Whatman #1 filter paper, and dried at 60° C. in an oven for 4 hours.

Results

The large particles settle faster than the small particles, enabling a size separation to be achieved. By removing the material from the bottom of the column at certain time points fractions with different size distributions were obtained.

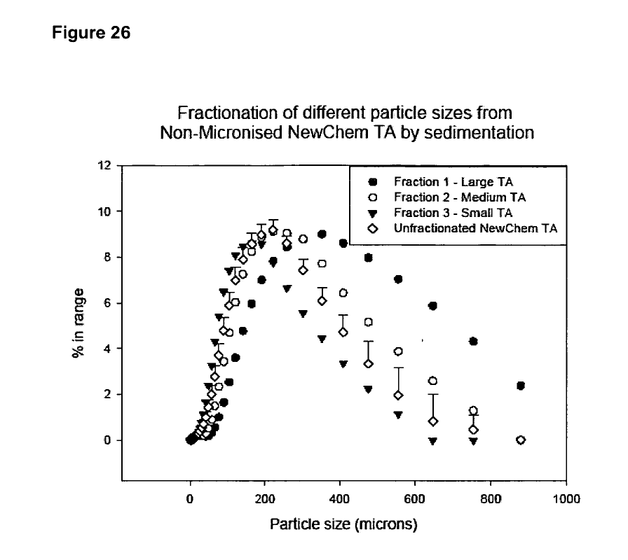

A separation of the NewChem TA material into different size fractions was achieved, and the particle size of the various fractions is illustrated in Table 7 below and FIG. 26. There is a clear separation achieved, evident on the D(v,0.9) values tabulated below; visually the samples looked very different and reflected the sizing results.

TABLE 7

| Material | D(v, 0.9%) μm |
|---|---|
| Unfractionated | 324 |
| Small fraction | 312 |
| Medium fraction | 444 |
| Large fraction | 591 |

### Example 15

Particle Size Reduction by Homogenization

Method

To reduce the particle size of TA to significantly lower than that obtained for the small fraction by sedimentation, a suspension of TA (20 mg/mL in suspending solution) was added to a 4 mL tube, and the shaft of a rotor-stator Polytron homogenizer immersed in the suspension prior to commencing the refinement.

Results

The homogenizer essentially snapped into fragments the large particles that were caught between the rotor and stator. Crystals of <200 μm were desired. However, it was desired to avoid producing too many fine particles, as this would produce unfavourable dissolution properties. Table 8 below shows the change in particle characteristics with homogenizer speed and exposure time.

TABLE 8

| Batch # | Speed | Time (secs) | D(v, 0.9) μm | Peak size^{a} (μm) |
|---|---|---|---|---|
| RGA0703 | — | 0 | 324 | 222 |
| RGA1501 | 3 | 5 | 341 | 222 |
| RGA1502 | 3 | 10 | 287 | 163 |
| RGA1503 | 3 | 20 | 312 | 191 |
| RGA1504 | 5 | 30 | 257 | 163 |
| RGA1505 | 6 | 60 | 208 | 141 |
| RGA1506 | 6 | 60 | 195 | 121 |

a Peak size is the size of the largest number of particles, i.e. the maximum in the % vs size plot.

The refinement of the particles was successful in bringing the D(v,0.9) down to approximately 200 μm using one minute refinement time, on maximum speed setting of 6.

### Example 16

Syringability

Method

To aid syringability, a TA solution was made up by combining 160 mg of non-micronised TA, 40 mg of micronised TA and 5 mL saline (0.9% NaCl in MilliQ H₂O) with stirring. The suspended TA was briefly sonicated in a Branson 220 sonicator bath (25° C., 50-60 Hz, 125 W) for 30 sec and returned to the stirrer.

50 mg of hyaluronic acid (HA) was weighed out and approximately 1/3 of this HA was added to the suspension. After stirring for 30 sec, the suspension was sonicated for 30 sec, then returned to the stirrer. The steps of adding HA, stirring and sonication were repeated until all HA had been added. The suspension was mixed for a further 30 min until all HA was dissolved. The suspension can be delivered through a 23 gauge needle.

The short periods of sonication (eg 30 sec) were used to break up weak crystal composites without substantially fracturing crystals.

### Example 17

In Vivo Studies

A 20/80 mixture of 400 μg micronised/non-micronised TA is injected into the eyes of rabbits. Each animal is its own control, as TA is injected into one eye and the other eye serves as the control.

Samples are withdrawn from the anterior chamber at regular intervals. The concentration of TA in the anterior chamber is correlated with the concentration of TA in the vitreous. The mechanism of passage of TA from the vitreous to the anterior chamber is one of simple diffusion and is a recognised method of assessment (Beer, et al. Intraocular concentrations and pharmacokinetics of TA after a single intravitreal injection, Ophthalmology 2003; 110:681-686). Vitreal samples are taken at regular intervals. All samples are assessed for TA content by HPLC. After the study period is complete, animals are sacrificed and ocular tissue samples collected.

Whilst the dose administered is lower than the therapeutic dose, efficacy has been shown at this lower dose.

### Example 18

In Vivo—Micronised vs Non-Micronised TA

Method

Twenty-five New Zealand albino rabbits weighing between 2-3 kg were used for this study. The animals were treated in accordance with the Association for Research in Vision and Ophthalmology resolution on the care and treatment of animals used in research.

Before all examinations and procedures, the animals were anesthetized with approximately 1 ml of a mixture of ketamine hydrochloride (50 mg/kg) and xylazine hydrochloride (5 mg/kg). Topical anaesthesia was achieved with the topical application of 0.5% proparacaine.

Slit-lamp and indirect funduscopic examinations were performed on all eyes before and after the drugs were administered and on days 1, 2, 3, 6, 9, 13, 16, 20, and 21.

The rabbits were divided into five groups. Group 1 (n=5) received topical 400 μg/0.1 ml TA (twice per day); Group 2 (n=5) received an intravitreal injection of 400 μg/0.1 ml of TA; Group 3 (n=5) underwent subconjunctival injection of 400 μg/0.1 ml of TA; Group 4 (n=5) were given an intravitreal injection of 400 μg/0.1 ml micronised TA; and Group 5 (n=5) received 400 μg/0.1 ml micronised TA subconjunctivally.

All procedures were performed under sterile conditions using an operating microscope for visualization.

Topical Application

The eyes in Group 1 received one drop of 400 μg TA topically twice daily during the study period. The drug was administered to the eyes as a single 50 μL drop.

Intravitreal Injections

The eyes in Groups 2 and 4 were injected intravitreally. An anterior chamber tap was performed to reduce intraocular pressure and to minimize drug reflux following injection. The intravitreal injection was performed using a 23-gauge needle attached to a tuberculin syringe inserted (bevel up) approximately 2 mm posterior to the limbus.

Subconjunctival Injections

The eyes in Groups 3 and 5 were given a subconjunctival injection using a 23-gauge needle attached to a tuberculin syringe inserted into the posterior-temporal conjunctiva. A cotton-tipped applicator was pressed against the area to minimize drug reflux following injection.

Anterior Chamber Paracentesis

An anterior chamber paracentesis (0.1 ml) was performed on days 1, 2, 3, 6, 9, 13, 16, and 20 after topical application, or subconjunctival or intravitreal injection in all groups. A 27-gauge 0.5-inch needle on a tuberculin syringe was inserted at the paralimbal clear cornea in a plane above and parallel to the iris, with the bevel facing forward, until the entire bevel penetrated the cornea. A 0.2 ml sample of fluid was withdrawn. The samples of aqueous fluid were immediately refrigerated at −70° C.

Euthanasia and Enucleation

The animals were sacrificed 21 days after the first treatment with an intravenous injection of 100 mg/kg sodium pentobarbital. The eyes were enucleated and placed in a −70° C. freezer.

Dissection of Eyes

All frozen eyes were dissected into 5 parts (the cornea, aqueous fluid, retina, sclera, and iris-corpus ciliare). The sclera in Groups 3 and 5 (subconjunctival injection) was dissected into two parts: nasal and temporal. The tissue was prepared for light microscopy.

### Example 19

In Vivo—Oedema Model

Vascular oedema is induced in New Zealand White/Dutch-belted rabbits. The left eye of each rabbit is injected with an isotonic 20/80 mixture of 400 μg micronised/non-micronised TA at physiological pH and constitutes the test eye. The right eye of each animal is considered the control, and is injected with Kenalog® A 40.

Effects of the TA on oedema are monitored with injected dye (indocyanine/fluorescein angiography) and further investigations with Optical Coherence Tomography (OCT).

Intraocular pressure is monitored twice daily for the first week, then daily, for a period of three months. If elevated intra-ocular pressure is still observed after this time, intraocular pressure may be monitored for six months or longer as required.

Toxicology studies are performed non-invasively during the study using an electroretinogram. Two weeks after injection, two rabbits are sacrificed for histopathology, then two rabbits every two weeks for the duration of the study.

### Example 20

In Vivo—Neovascularisation Model

Neovascularisation is induced in New Zealand White/Dutch-belted rabbits with laser burn. The left eye of each rabbit is injected with an isotonic 20/80 mixture of 400 ug micronised/non-micronised TA at physiological pH and constitutes the test eye. The right eye of each animal is considered the control, and is injected with Kenalog® A 40.

Neovascularisation is monitored with slit-lamp biomicroscopy and fundus photography.

Intraocular pressure is monitored twice daily for the first week, then daily, for a period of six months.

Toxicology studies are conducted non-invasively during the study using an electroretinogram.

Two weeks after injection, two rabbits are sacrificed for histopathology, then two rabbits every two weeks for the duration of the study.

Although the invention has been described with reference to certain preferred embodiments, it will be appreciated that many variations and modifications may be made within the scope of the broad principles of the invention. Hence, it is intended that the preferred embodiments and all of such variations and modifications be included within the scope and spirit of the invention, as defined by the following claims.

The invention is further described by the following numbered paragraphs:
1. A pharmaceutically acceptable composition comprising an anti-inflammatory steroid or pharmaceutically acceptable salt thereof, wherein the steroid or pharmaceutically acceptable salt thereof is formed of crystals or crystal composites, and wherein the pharmaceutically acceptable composition comprises a greater proportion of crystals and crystal composites having a diameter greater than about 20 μm than crystals and crystal composites having a diameter less than about 20 μm.
2. The pharmaceutically acceptable composition of paragraph 1, wherein the composition comprises crystals with diameters in the range of about 50 μm to 600 μm.
3. The composition of paragraph 2 wherein the proportion of the crystals is greater than the proportion of crystal composites with diameters in the range of about 50 μm to 600 μm.
4. A pharmaceutically acceptable composition comprising an anti-inflammatory steroid or pharmaceutically acceptable salt thereof, wherein the steroid or pharmaceutical acceptable salt thereof formed as crystals or crystal composites, and wherein the crystals are further comprised of a first set of crystals that range in diameter from about 0.5 um to about 40 um and a second set of crystals that range in size from about 50 um to about 600 um.
5. The composition of paragraph 4 wherein the first set of crystals are more concentrated than the crystal composites.
6. The composition of paragraph 4 wherein the first set of crystals range in diameter from about 1 μm to about 40 μm, about 5 μm to about 35 μm, about 10 μm to about 30 μm, about 15 μm to about 25 μm or about 20 μm to about 22 μm.
7. The composition of paragraph 4 wherein the second set of crystals range in diameter from about 70 μm to about 400 μm, about 80 μm to about 300 μm, about 90 μm to about 250 μm or about 100 μm to about 200 μm.
8. The composition of any one of paragraphs 1 to 7 wherein the anti-inflammatory steroid is a 11-substituted-16α,17α-substituted methylenedioxy steroid of the formula: 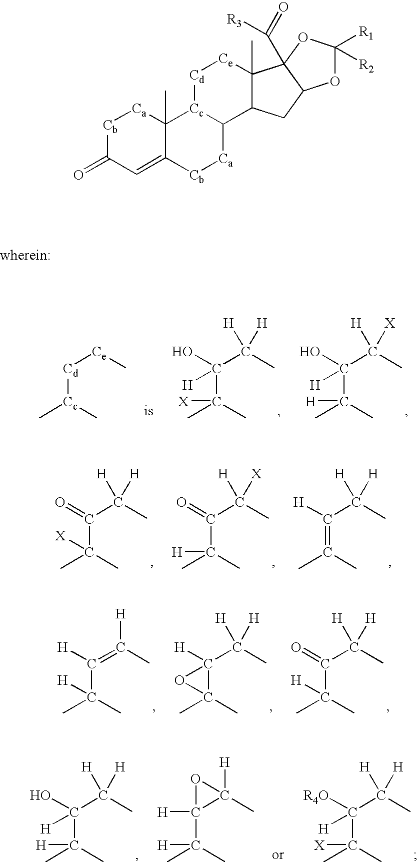
   R₁ and R₂ are hydrogen or alkyl; 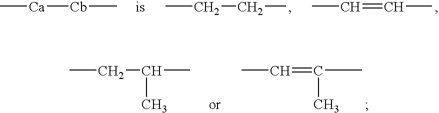
   R₃ is methyl, hydroxymethyl or methylaminoalkylenecarbonyloxymethyl, alkylcarbonyloxymethyl, or phenylaminoalkylenecarbonyloxymethyl;
   R₄ is alkanoyl; and
   X is a halogen.
9. The composition of any one of paragraphs 1 to 7 wherein the compound is of the formula: 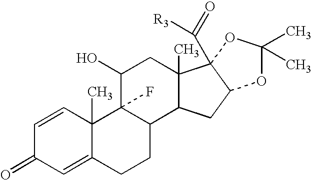
   wherein R₃ is hydroxymethyl, phenylcarbonylaminoisopropylcarbonyloxymethyl, or 2,2-dimethylpropylcarbonyloxymethyl.
10. The composition of any one of paragraphs 1 to 7 wherein the compound is crystalline 9-fluoro-11,21-dihydroxy-16,17-[1-methylethylidinebis(oxy)]pregna-1,4-diene-3,20-dione: 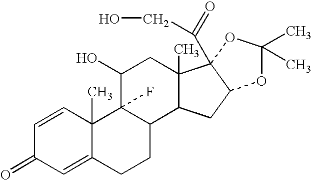
11. The compound of any one of paragraphs 8 to 10 which is a pharmaceutically acceptable salt.
12. The composition of any one of paragraphs 4 to 11 wherein the weight per volume ratio of the first set of crystals to the second set of crystals is about 1:1, 1:2, 2:1, 1:3, 3:1, 2:3, 3:2, 1:4, 4:1, 3:4, 4:3, 1:5, 5:1, 2:5, 5:2, 3:5, 5:3, 4:5, 5:4, 1:6, 6:1, 5:6, or 6:5.
13. The composition of any one of paragraphs 1 to 11 wherein the composition comprises about 20% w/v of crystals of about 0.5 μm to about 40 μm and 80% w/v of crystals of about 50 μm to about 600 μm.
14. The composition of any one of paragraphs 1 to 11 wherein the composition comprises about 25% w/v of crystals of about 0.5 μm to about 40 μm and about 75% w/v of crystals of about 50 μm to about 600 μm.
15. The composition of any one of paragraphs 1 to 11 wherein the composition comprises about 50% w/v of crystals of about 0.5 μm to about 40 μm and about 50% w/v of crystals of about 50 μm to about 600 μm.
16. The composition of any one of paragraphs 1 to 11 wherein the composition comprises about 75% w/v of crystals of about 0.5 μm to about 40 μm and about 25% w/v of crystals of about 50 μm to about 600 μm.
17. The composition of any one of paragraphs 1 to 11 wherein the composition comprises about 20% w/v of crystals of about 0.5 μm to about 40 μm and about 80% w/v of crystals of about 100 μm to about 200 μm.
18. The composition of any one of paragraphs 1 to 11 wherein the composition comprises about 25% w/v of crystals of about 0.5 μm to about 40 μm and about 75% w/v of crystals of about 100 μm to about 200 μm.
19. The composition of any one of paragraphs 1 to 11 wherein the composition comprises about 50% w/v of crystals of about 0.5 μm to about 40 μm and about 50% w/v of crystals of about 100 μm to about 200 μm
20. The composition of any one of paragraphs 1 to 11 wherein the composition comprises about 75% w/v of crystals of about 0.5 μm to about 40 μm and about 25% w/v of crystals of about 100 μm to about 200 μm.
21. A method of preparing a pharmaceutically acceptable triamcinolone acetonide composition which has an improved therapeutically effective dwell time in the vitreous in a patient, the composition comprising crystals and crystal composites of triamcinolone acetonide, wherein the method comprises increasing the concentration of the crystals, as compared to the concentration of the crystal composites, in the composition.
22. A method of preparing a pharmaceutically acceptable triamcinolone acetonide composition which has an improved therapeutically effective dwell time in the vitreous in a patient, the composition comprising crystals and crystal composites of triamcinolone acetonide, wherein the method comprises increasing the proportion of the crystals compared to the proportion of crystal composites in the composition, wherein the crystals and crystal composites have diameters ranging from about 50 μm to about 600 μm.
23. A method of preparing a pharmaceutically acceptable triamcinolone composition which has an improved therapeutically effective dwell time in the vitreous in a patient, said method comprising selecting triamcinolone crystals with diameters in the range of about 50 μm to about 600 μm from another triamcinolone composition comprising both crystals and crystal composites.
24. The method of any one of paragraphs 21 to 23 comprising the additional steps of: selecting triamcinolone crystals in the size range of about 100 μm to about 200 μm from a triamcinolone composition comprising both crystals and crystal composites.
25. The method of any one of paragraphs 21 to 24 comprising the additional steps of: adding said range of crystals to an ophthalmologically acceptable carrier, diluent and/or excipient.
26. A pharmaceutically acceptable composition prepared according to the method of any one of paragraphs 21 to 25.
27. The composition of any one of paragraphs 1 to 11 additionally comprising at least one pharmaceutically acceptable additive.
28. The composition of paragraph 27 wherein the additive is ophthalmologically acceptable.
29. The composition of paragraph 27 wherein the additive is compatible with the vitreous and does not leave any vision impairing residue in the eye.
30. The composition of paragraph 27 wherein the additive is suited to the delivery of said pharmaceutical composition as an intravitreal depot injection.
31. The composition of paragraph 27 wherein the additive is a diluent.
32. The composition of paragraph 31 wherein the diluent is selected from the group comprising: water, a saline salt solution, an organic salt solution, an inorganic salt solutions, Ringer's solution, dextrose solution, and Hank's solution.
33. The composition of paragraph 31 wherein the diluent is a balanced salt solution.
34. The composition of paragraphs 33 wherein the balanced salt solution is Ringer's lactate medium.
35. A method of treating an inflammatory eye condition in a patient in need thereof, said method comprising administering to or adjacent to at least an ocular tissue a pharmaceutically acceptable composition according to any one of paragraphs 1 to 20 or a pharmaceutically acceptable composition prepared by the method according to any one of paragraphs 21 to 25 or a pharmaceutically acceptable composition according to any one of paragraphs 26 to 34.
36. The method of paragraph 35 wherein the composition is administered by topical application, cannular delivery, periorbital injection into the orbital floor, sub-conjunctival injection, implantation within the eye with or without suturing or intraocular injection.
37. The method of paragraph 36 wherein the intraocular injection is an intravitreal injection, aqueous humour injection or injection into the external layers of the eye, such as subconjunctival injection or sub-Tenon injection.
38. The method of paragraph 36 wherein the intraocular injection is carried out via a self sealing 21-30 gauge needle or other suitably calibrated delivery device through the pars plana.
39. The method of paragraph 36 wherein the topical application is by ointment, gel or eye drops.
40. The method of paragraph 35 wherein the pharmaceutically acceptable composition is delivered at a concentration sufficient to achieve a final concentration of the pharmaceutically acceptable composition within the target ocular compartment between about 0.05 mg/ml and about 25 mg/ml.
41. The method of paragraph 35 wherein the pharmaceutically acceptable composition is administered by intraocular delivery and the final concentration of the pharmaceutically acceptable composition is between about 0.05 mg/ml and about 8 mg/ml.
42. The method of paragraph 41 wherein the concentration is between about 1 mg/ml and about 7 mg/ml, between about 1.5 mg/ml and about 6 mg/ml, between about 2 mg/ml and about 5 mg/ml, or between about 3 mg/ml and about 4 mg/ml.
43. The method of paragraph 40 wherein the pharmaceutically acceptable composition is administered intravitreally and the final concentration of the pharmaceutically acceptable composition compound is about 4 mg/ml.
44. The composition of any one of paragraphs 1 to 11 wherein the compositions are administered in unit dosage forms suitable for single administration of precise dosage amounts.
45. The method of paragraph 35 wherein the composition is administered every 1 to 3 months.
46. The method of paragraph 35 wherein the composition is administered less frequent than every 3 months.
47. A pharmaceutically acceptable composition of an anti-inflammatory steroid or pharmaceutically acceptable salt thereof, wherein the anti-inflammatory steroid or pharmaceutically acceptable salt thereof is formed of crystals, and wherein the crystals further comprise a first set of crystals with diameters ranging from about 0.5 μm to about 40 μm and a second set of crystals with diameters from about 50 μm to about 600 μm, and wherein the pharmaceutically acceptable composition further comprises a biocompatible, biodegradable matrix.
48. The method of paragraph 35 wherein administration of the pharmaceutically acceptable composition is performed in combination with one or more other therapies such as photodynamic therapy, laser treatment, or one or more biological or pharmaceutical treatments.
49. The method of paragraph 48 wherein the other therapy is laser treatment of the retina and administration of an anti-inflammatory steroid is carried out by injection before or after the laser treatment.
50. The method of paragraph 35 wherein at least one additional compound is administered with the pharmaceutically acceptable composition, said additional compound selected from the group consisting of: antibiotics, anti-angiogenesis agents, glucocorticoids (e.g. prednisolone, prednisone), oestrogens (e.g. oestrodiol), androgens (e.g. testosterone) retinoic acid derivatives (e.g. 9-cis-retinoic acid, 13-trans-retinoic acid, all-trans retinoic acid), vitamin D derivatives (e.g. calcipotriol, calcipotriene), non-steroidal anti-inflammatory agents, anti-infective agent, protein kinase C inhibitors, MAP kinase inhibitors, anti-apoptotic agents, growth factors, vitamins, and unsaturated fatty acids.
51. The method according to paragraph 50 wherein the anti-angiogenic agent is Lucentis® or Macugen®.
52. A pharmaceutically acceptable composition according to any one of paragraphs 1 to 20 or a pharmaceutically acceptable composition prepared by the method according to any one of paragraphs 21 to 25 or a pharmaceutically acceptable composition according to any one of paragraphs 26 to 34 wherein the composition also comprises at least one additional compound selected from the group consisting of: antibiotics, anti-angiogenesis agents, glucocorticoids (e.g. prednisolone, prednisone), oestrogens (e.g. oestrodiol), androgens (e.g. testosterone) retinoic acid derivatives (e.g. 9-cis-retinoic acid, 13-trans-retinoic acid, all-trans retinoic acid), vitamin D derivatives (e.g. calcipotriol, calcipotriene), non-steroidal anti-inflammatory agents, anti-infective agent, protein kinase C inhibitors, MAP kinase inhibitors, anti-apoptotic agents, growth factors, vitamins, and unsaturated fatty acids.
53. A pharmaceutically acceptable composition according to paragraph 52 wherein the anti-angiogenic agent is selected from the group consisting of Lucentis® and Macugen®.

1. A pharmaceutically acceptable composition comprising an anti-inflammatory steroid or pharmaceutically acceptable salt thereof, wherein the steroid or pharmaceutically acceptable salt thereof is formed of crystals or crystal composites, and wherein the pharmaceutically acceptable composition comprises a greater proportion of crystals and crystal composites having a diameter greater than about 20 μm than crystals and crystal composites having a diameter less than about 20 μm or
wherein the composition comprises crystals with diameters in the range of about 50 μm to 600 μm or
wherein the proportion of the crystals is greater than the proportion of crystal composites with diameters in the range of about 50 μm to 600 μm.

2. A pharmaceutically acceptable composition comprising an anti-inflammatory steroid or pharmaceutically acceptable salt thereof, wherein the steroid or pharmaceutical acceptable salt thereof formed as crystals or crystal composites, and wherein the crystals are further comprised of a first set of crystals that range in diameter from about 0.5 um to about 40 um and a second set of crystals that range in size from about 50 um to about 600 um or
wherein the first set of crystals are more concentrated than the crystal composites or
wherein the first set of crystals range in diameter from about 1 μm to about 40 μm, about 5 μm to about 35 μm, about 10 μm to about 30 μm, about 15 μm to about 25 μm or about 20 μm to about 22 μm.

3. The composition of claim 2 wherein the second set of crystals range in diameter from about 70 μm to about 400 μm, about 80 μm to about 300 μm, about 90 μm to about 250 μm or about 100 μm to about 200 μm.

4. The composition of claim 1 wherein the anti-inflammatory steroid is a 11-substituted-16α,17α-substituted methylenedioxy steroid of the formula: 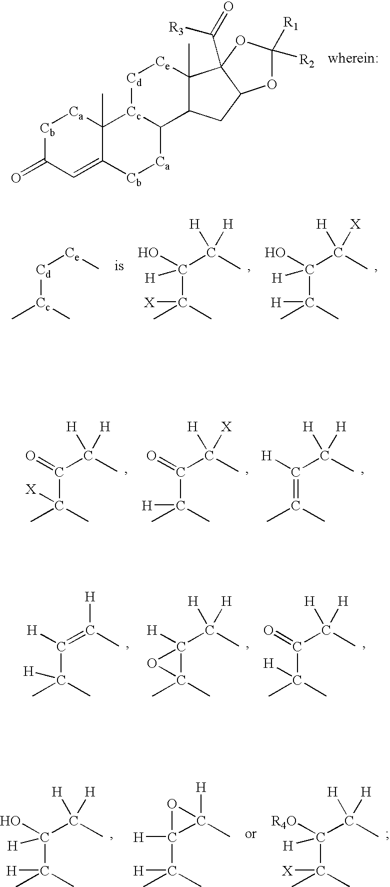
R₁ and R₂ are hydrogen or alkyl;
—Ca—Cb—is
—CH₂—CH₂—, —CH═CH—, 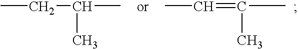
R₃ is methyl, hydroxymethyl or methylaminoalkylenecarbonyloxymethyl, alkylcarbonyloxymethyl, or phenylaminoalkylenecarbonyloxymethyl;
R₄ is alkanoyl; and
X is a halogen.

5. The composition of claim 1 wherein the steroid or pharmaceutically acceptable salt thereof is of the formula: 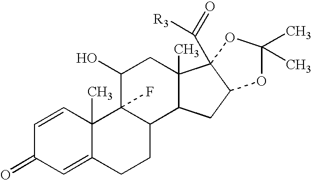
wherein R₃ is hydroxymethyl, phenylcarbonylaminoisopropylcarbonyloxymethyl, or 2,2-dimethylpropylcarbonyloxymethyl.

6. The composition of claim 1 wherein the steroid or pharmaceutically acceptable salt thereof is crystalline 9-fluoro-11,21-dihydroxy-16,17-[1-methylethylidinebis(oxy)]pregna-1,4-diene-3,20-dione:

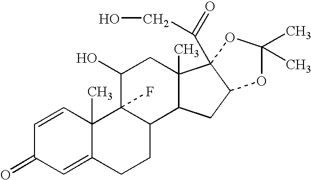

7. The composition of claim 4 wherein the anti-inflammatory steroid is a pharmaceutically acceptable salt.

8. The composition of claim 2 wherein the weight per volume ratio of the first set of crystals to the second set of crystals is about 1:1, 1:2, 2:1, 1:3, 3:1, 2:3, 3:2, 1:4, 4:1, 3:4, 4:3, 1:5, 5:1, 2:5, 5:2, 3:5, 5:3, 4:5, 5:4, 1:6, 6:1, 5:6, or 6:5.

9. The composition of claim 1 wherein the composition comprises about 20% w/v of crystals of about 0.5 μm to about 40 μm and 80% w/v of crystals of about 50 μm to about 600 μm or
wherein the composition comprises about 25% w/v of crystals of about 0.5 μm to about 40 μm and about 75% w/v of crystals of about 50 μm to about 600 μm or
wherein the composition comprises about 50% w/v of crystals of about 0.5 μm to about 40 μm and about 50% w/v of crystals of about 50 μm to about 600 μm or
wherein the composition comprises about 75% w/v of crystals of about 0.5 μm to about 40 μm and about 25% w/v of crystals of about 50 μm to about 600 μm or
wherein the composition comprises about 20% w/v of crystals of about 0.5 μm to about 40 μm and about 80% w/v of crystals of about 100 μm to about 200 μm or
wherein the composition comprises about 20% w/v of crystals of about 0.5 μm to about 40 μm and about 80% w/v of crystals of about 100 μm to about 200 μm or
wherein the composition comprises about 25% w/v of crystals of about 0.5 μm to about 40 μm and about 75% w/v of crystals of about 100 μm to about 200 μm or
wherein the composition comprises about 50% w/v of crystals of about 0.5 μm to about 40 μm and about 50% w/v of crystals of about 100 μm to about 200 μm or
wherein the composition comprises about 75% w/v of crystals of about 0.5 μm to about 40 μm and about 25% w/v of crystals of about 100 μm to about 200 μm.

10. A method of preparing a pharmaceutically acceptable triamcinolone acetonide composition which has an improved therapeutically effective dwell time in the vitreous in a patient, the composition comprising crystals and crystal composites of triamcinolone acetonide, wherein the method comprises increasing the concentration of the crystals, as compared to the concentration of the crystal composites, in the composition.

11. A method of preparing a pharmaceutically acceptable triamcinolone acetonide composition which has an improved therapeutically effective dwell time in the vitreous in a patient, the composition comprising crystals and crystal composites of triamcinolone acetonide, wherein the method comprises increasing the proportion of the crystals compared to the proportion of crystal composites in the composition, wherein the crystals and crystal composites have diameters ranging from about 50 μm to about 600 μm or a method of preparing a pharmaceutically acceptable triamcinolone composition which has an improved therapeutically effective dwell time in the vitreous in a patient, said method comprising selecting triamcinolone crystals with diameters in the range of about 50 μm to about 600 μm from another triamcinolone composition comprising both crystals and crystal composites.

12. The method of claim 10 comprising the additional steps of: selecting triamcinolone crystals in the size range of about 100 μm to about 200 μm from a triamcinolone composition comprising both crystals and crystal composites or adding said range of crystals to an ophthalmologically acceptable carrier, diluent and/or excipient.

13. A pharmaceutically acceptable composition prepared according to the method of claim 10.

14. The composition of claim 1 additionally comprising at least one pharmaceutically acceptable additive,
wherein the additive is ophthalmologically acceptable or
wherein the additive is compatible with the vitreous and does not leave any vision impairing residue in the eye or
wherein the additive is suited to the delivery of said pharmaceutical composition as an intravitreal depot injection or
wherein the additive is a diluent or
wherein the diluent is selected from the group comprising: water, a saline salt solution, an organic salt solution, an inorganic salt solutions, Ringer's solution, dextrose solution, and Hank's solution or
wherein the diluent is a balanced salt solution or
wherein the balanced salt solution is Ringer's lactate medium.

15. A method of treating an inflammatory eye condition in a patient in need thereof, said method comprising administering to or adjacent to at least an ocular tissue a pharmaceutically acceptable composition according to claim 1,
wherein the composition is administered by topical application, cannular delivery, periorbital injection into the orbital floor, sub-conjunctival injection, implantation within the eye with or without suturing or intraocular injection or
wherein the intraocular injection is an intravitreal injection, aqueous humour injection or injection into the external layers of the eye, such as subconjunctival injection or sub-Tenon injection or
wherein the intraocular injection is carried out via a self sealing 21-30 gauge needle or other suitably calibrated delivery device through the pars plana or
wherein the topical application is by ointment, gel or eye drops or
wherein the pharmaceutically acceptable composition is delivered at a concentration sufficient to achieve a final concentration of the pharmaceutically acceptable composition within the target ocular compartment between about 0.05 mg/ml and about 25 mg/ml or
wherein the pharmaceutically acceptable composition is administered by intraocular delivery and the final concentration of the pharmaceutically acceptable composition is between about 0.05 mg/ml and about 8 mg/ml or
wherein the concentration is between about 1 mg/ml and about 7 mg/ml, between about 1.5 mg/ml and about 6 mg/ml, between about 2 mg/ml and about 5 mg/ml, or between about 3 mg/ml and about 4 mg/ml or
wherein the pharmaceutically acceptable composition is administered intravitreally and the final concentration of the pharmaceutically acceptable composition compound is about 4 mg/ml or
wherein the composition is administered every 1 to 3 months or
wherein the composition is administered less frequent than every 3 months or
wherein administration of the pharmaceutically acceptable composition is performed in combination with one or more other therapies such as photodynamic therapy, laser treatment, or one or more biological or pharmaceutical treatments or
wherein the other therapy is laser treatment of the retina and administration of an anti-inflammatory steroid is carried out by injection before or after the laser treatment or
wherein at least one additional compound is administered with the pharmaceutically acceptable composition, said additional compound selected from the group consisting of: antibiotics, anti-angiogenesis agents, glucocorticoids (e.g. prednisolone, prednisone), oestrogens (e.g. oestrodiol), androgens (e.g. testosterone) retinoic acid derivatives (e.g. 9-cis-retinoic acid, 13-trans-retinoic acid, all-trans retinoic acid), vitamin D derivatives (e.g. calcipotriol, calcipotriene), non-steroidal anti-inflammatory agents, anti-infective agent, protein kinase C inhibitors, MAP kinase inhibitors, anti-apoptotic agents, growth factors, vitamins, and unsaturated fatty acids or
wherein the anti-angiogenic agent is Lucentis® or Macugen®.

16. The composition of claim 1 wherein the composition is administered in unit dosage forms suitable for single administration of precise dosage amounts.

17. A pharmaceutically acceptable composition of an anti-inflammatory steroid or pharmaceutically acceptable salt thereof, wherein the anti-inflammatory steroid or pharmaceutically acceptable salt thereof is formed of crystals, and wherein the crystals further comprise a first set of crystals with diameters ranging from about 0.5 μm to about 40 μm and a second set of crystals with diameters from about 50 μm to about 600 μm, and wherein the pharmaceutically acceptable composition further comprises a biocompatible, biodegradable matrix.

18. A pharmaceutically acceptable composition according to claim 1 wherein the composition also comprises at least one additional compound selected from the group consisting of: antibiotics, anti-angiogenesis agents, glucocorticoids (e.g. prednisolone, prednisone), oestrogens (e.g. oestrodiol), androgens (e.g. testosterone) retinoic acid derivatives (e.g. 9-cis-retinoic acid, 13-trans-retinoic acid, all-trans retinoic acid), vitamin D derivatives (e.g. calcipotriol, calcipotriene), non-steroidal anti-inflammatory agents, anti-infective agent, protein kinase C inhibitors, MAP kinase inhibitors, anti-apoptotic agents, growth factors, vitamins, and unsaturated fatty acids and wherein the anti-angiogenic agent is selected from the group consisting of Lucentis® and Macugen®.
